(19) United States
(12) Patent Application Publication
(10) Pub. No.: US 2006/0263427 A1
(43) Pub. Date: Nov. 23, 2006

(54) Quinine formulations

(76) Inventors: Richard Howard Roberts, Lakewood, NJ (US); Kristin Arnold, Morrisville, PA (US); Jie Du, Lansdale, PA (US)

Correspondence Address: CANTOR COLBURN, LLP, 55 GRIFFIN ROAD SOUTH, BLOOMFIELD, CT, 06002, US

(21) Appl. No.: 11/415,940

(22) Filed: May 2, 2006

Related U.S. Application Data
(60) Provisional application No. 60/729,574, filed on Oct. 24, 2005.
(60) Provisional application No. 60/677,269, filed on May 3, 2005.

(51) Int. Cl. A61K 31/44; A61K 9/22; A61K 31/4745

(52) U.S. Cl. 424/468; 514/305

(57) ABSTRACT

Disclosed herein are controlled-release quinine formulations and methods of preparing the same. Also disclosed are methods of preventing or treating malaria, leg cramps, or babesiosis by administering the controlled-release quinine formulations. The controlled-release quinine formulations may help to reduce or eliminate adverse side effects typically associated with the dosing of quinine.

### CROSS-REFERENCE TO RELATED APPLICATIONS

This application claims the benefit of U.S. Provisional Application Ser. No. 60/729,574, filed Oct. 24, 2005 and U.S. Provisional Application Ser. No. 60/677,269, filed May 3, 2005, both of which are incorporated by reference in their entirety.

### BACKGROUND

Malaria is a parasitic disease caused by the Plasmodium species P. falciparum, P. vivax, P. ovale and P. malariae. The malaria parasite causes intermittent fevers and chills. It affects multiple organs and systems, including red blood cells, the kidneys, liver, spleen and brain. It is estimated by the World Health Organization (WHO) that up to 500 million persons per year are infected with malaria, with 200 to 300 million people suffering from malaria at any given time (See Roll Back Malaria. World Health Organization. available at: www.rbm.who.int/cmc_upload/0/000/015372/RBMInfosheet_{—}1.htm). Up to 3 million will die each year. If P. falciparum infection goes untreated or is not treated appropriately, general observations indicate that mortality is high, killing up to 25% of non-immune adults within 2 weeks of a primary attack [Taylor T E, Strickland G T. Malaria. In: Strickland G T, ed. Hunter's Tropical Medicine and Emerging Infectious Diseases. 8th ed. Philadelphia, Pa.: W.B. Saunders Company; 2000.] A significant number of these cases are found in Central America, South America, Asia, and Africa. Known antimalarial agents include 9-aminoacridines (e.g. mepacrine), 4-aminoquinolines (e.g. amodiaquine, chloroquine, hydroxychloroquine), 8-aminoquinolines (e.g. primaquine, quinocide), biguanides with an inhibiting effect on dihydrofolic acid reductase (e.g. chlorproguanil, cycloguanil, proguanil), diaminopyrimidines (e.g. pyrimethamine), quinine salts, sulphones such as dapsone, sulphonamides, sulphanilamides and antibiotics such as tetracycline.

Quinine (cinchonan-9-ol, 6′-methoxy-, (8α,9R)-) is an antiprotozoal and an antimyotonic, and is known for the treatment of malaria caused by Plasmodium species, the treatment and prophylaxis of nocturnal recumbency leg muscle cramps, and the treatment of babesiosis caused by Babesia microti. Quinine is structurally similar to quinidine, which is also an antiprotozoal, but can function as an antiarrhythmic. Quinidine has been associated with the prolongation of the QT interval in a dose-related fashion. Prolongation of the electrocardiographic QT interval can be indicative of delayed ventricular repolarization. Excessive QT prolongation has been associated with an increased risk of ventricular arrhythmia. Although quinine is a diastereomer of quinidine, it does not cause QT prolongation to the same degree although it has been suggested that patients with a history of cardiac arrhythmias or QT prolongation should carefully consider taking quinine as they may be at risk for arrhythmias.

There remains a need in the art for quinine formulations that provide a desired therapeutic effect against certain diseases (e.g., malaria) while at the same time minimizing the adverse side effects associated with dosing of quinine.

### SUMMARY

Disclosed herein are controlled-release quinine and quinine combination formulations, as well as methods of using such controlled-release formulations for therapeutic purposes. Exemplary therapeutic purposes include the treatment or prevention malaria; leg cramps including nocturnal recumbency leg muscle cramps, idiopathic leg cramps, and leg cramps caused by athletic exertion; and babesiosis caused by Babesia microti.

In one embodiment, a controlled-release formulation comprises a therapeutically effective amount of quinine; wherein dosing of the controlled-release formulation results in reducing or eliminating an adverse side effect associated with dosing of an immediate-release quinine formulation.

In another embodiment, a method of reducing the severity or eliminating an adverse side effect associated with the administration of an immediate-release quinine formulation comprises administering to a patient a controlled-release quinine formulation.

### BRIEF DESCRIPTION OF THE DRAWINGS

FIG. 1 Mean plasma concentrations and QTc measurements over 24-hours following a single oral dose of Quinine Sulfate under fasting conditions;

FIG. 2 Mean plasma concentrations and QTc measurements over 24-hours following a single oral dose of Quinine Sulfate under fed conditions;

FIG. 3 FIG. 3 Mean plasma concentration and QTc measurements over 24-hours following a single oral dose of Quinine Sulfate 324 mg under fasting conditions;

FIG. 4 Mean plasma concentration and QTc measurements over 24-hours following a single oral dose of Quinine Sulfate 648 mg under fasting conditions.

### DETAILED DESCRIPTION

Quinine therapy can be considered optimal when effective plasma levels are reached when required. In addition, peak plasma values (Cₘₐₓ) should be as low as possible so as to reduce the incidence and severity of possible side effects, including the adverse event of QT prolongation. For the convenience of the patient or caretaker, a quinine dosage form that can be administered once daily and yields effective plasma levels for 8 to 24 hours would be desirable.

Controlled-release forms of quinine or its pharmaceutically acceptable salt may be found to provide a reduction in adverse side effects often associated with dosing immediate-release forms of quinine of the same dosage strength. Described herein are controlled-release quinine formulations, methods of preparing, and methods of use thereof.

The controlled-release quinine formulations may provide a decrease in adverse side effects that are associated with high doses of quinine, or even those associated with therapeutic doses. Such adverse side effects that can be mitigated include, for example, cinchonism, tinnitus, blurred vision, thrombocytopenia, granulomatous hepatitis, skin rash, acute interstitial nephritis, thrombotic thrombocytopenia purpura-hemolytic-uremic syndrome (TTP-HUS), QT interval prolongation, QTc interval prolongation, agranulocytosis, hypoprothrombinemia, disseminated intravascular coagulation, hemolytic anemia, hemolytic uremic syndrome, headache, diplopia, confusion, altered mental status, seizures, coma, pruritus, flushing of the skin, sweating, occasional edema of the face, exanthema, urticaria, erythema multiforme, purpura, photosensitivity, contact dermatitis, acral necrosis, cutaneous vasculitis, asthmatic symptoms, tachycardia, irregular rhythm, premature ventricular contractions (PVCs), nodal escape beats followed the PVCs, U waves with normal PR, QRS, and QT intervals, ventricular fibrillation, arrhythmia, nausea and vomiting, abdominal pain, diarrhea, visual disturbances, including sudden loss of vision, blindness, diminished visual fields, fixed papillary dilatation, disturbed color vision, hearing loss, and deafness.

As used herein, the controlled-release quinine formulation, as compared to immediate-release formulations (e.g. dosed TID), may provide reduction in the duration or magnitude of QT prolongation events as determined by surface electrocardiogram (EKG) measured from the beginning of the QRS complex to the end of the T wave, which represents the duration of activation and recovery of the ventricular myocardium. The QT values are heart rate corrected to “QTc”. Generally, a QTc above about 0.44 seconds is considered abnormal, although there are age- and sex-specific abnormal QTc values which vary from this number.

As used herein, the term “wherein dosing of the controlled-release formulation does not cause significant QT prolongation according to the standards of the United States Food and Drug Administration” means the standards found in the document Guidance for Industry, E14 Clinical Evaluation of QT/QTc Interval Prolongation and Proarrhythmic Potential for Non-Antiarrhythmic Drugs, U.S. Department of Health and Human Services Food and Drug Administration, Center for Drug Evaluation and Research (CDER), Center for Biologics Evaluation and Research (CBER) issued October 2005 and available at http://www.fda.gov/cder/guidance/index.htm.

The controlled-release formulations of quinine or its pharmaceutically acceptable salts are formulated to provide more consistent plasma levels of quinine and the active metabolite 3-hydroxyquinine than immediate-release forms. More consistent plasma levels may result in the reduction of the duration of QT or QTc interval prolongation that may otherwise be associated with increased doses or “dose dumping” of quinine. Furthermore, more consistent plasma levels may also result in the reduction or avoidance of other adverse side effects as outline previously.

An additional advantage to a controlled-release formulation, especially extended-release, is an increase in patient compliance and ease of dispensing for the pharmacist as there will be fewer dosage forms to count and package. Currently, immediate-release oral dosage tablets of quinine sulfate used to treat P. falciparum or babesiosis are commonly dosed at 600-650 mg every eight hours. By reducing the number of doses per day as well as potentially reducing or eliminating certain adverse side effects, patients would comply more strictly to prescribed dosing regimens. Increased compliance to the dosing regimen provides an increased chance of a successful treatment to the particular disease or disorder targeted.

Generally, suitable extended-release forms include wax or polymer coated tablets, caplets, or drug cores; time-release matrices; or a combination comprising at least one of the foregoing. Other dosage forms for oral administration include, for example, suspension, an emulsion, orally disintegrating tablets including effervescent tablets, chewable tablets, gastro-resistant tablets, soft capsules, hard capsules, gastro-resistant capsules, coated granules, gastro-resistant granules, modified-release granules, osmotic pumps, and the like. Examples of extended-release formulations which are suitable for use with quinine or salts thereof include those provided in Sustained Release Medications, Chemical Technology Review No. 177. Ed. J. C. Johnson. Noyes Data Corporation 1980; and Controlled Drug Delivery, Fundamentals and Applications, 2nd Edition. Eds. J. R. Robinson, V. H. L. Lee. Mercel Dekker Inc. New York 1987. Additional forms are described in U.S. Pat. Nos. 5,102,666 and 5,422,123.

An “active agent” means a compound, element, or mixture that when administered to a patient, alone or in combination with another compound, element, or mixture, confers, directly or indirectly, a physiological effect on the patient. The indirect physiological effect may occur via a metabolite or other indirect mechanism. When the active agent is a compound, then salts, solvates (including hydrates) of the free compound or salt, crystalline forms, non-crystalline forms, and any polymorphs of the compound are contemplated herein.

“Pharmaceutically acceptable salts” include derivatives of quinine, wherein the parent compound is modified by making non-toxic acid addition salts thereof, and further refers to pharmaceutically acceptable solvates, including hydrates, of such compounds and such salts. Also included are all crystalline, amorphous, and polymorph forms. Examples of pharmaceutically acceptable salts include, but are not limited to, mineral or organic acid addition salts; and the like, and a combination comprising at least one of the foregoing salts. The pharmaceutically acceptable salts include non-toxic salts, for example, from non-toxic inorganic or organic acids. For example, non-toxic acid salts include those derived from inorganic acids such as hydrochloric, hydrobromic, sulfuric, sulfamic, phosphoric, nitric and the like. Pharmaceutically acceptable organic salts includes salts prepared from organic acids such as acetic, trifluoroacetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like. Specific quinine salts include quinine sulfate, quinine hydrochloride, quinine dihydrochloride, and hydrates thereof.

“Quinine” as used herein is inclusive of all pharmaceutically acceptable salt forms, crystalline forms, amorphous form, polymorphic forms, solvates, and hydrates unless specifically indicated otherwise. As used herein, quinine sulfate means cinchonan-9-ol, 6′-methoxy-, (8α,9R)-, sulfate (2:1) or cinchonan-9-ol, 6′-methoxy-, (8α,9R)-, sulfate (2:1) dehydrate unless otherwise indicated.

“Bioavailability” means the extent or rate at which an active agent is absorbed into a living system or is made available at the site of physiological activity. For active agents that are intended to be absorbed into the bloodstream, bioavailability data for a given formulation may provide an estimate of the relative fraction of the administered dose that is absorbed into the systemic circulation. “Bioavailability” can be characterized by one or more pharmacokinetic parameters.

A “dosage form” means a unit of administration of an active agent. Examples of dosage forms include tablets, capsules, injections, suspensions, liquids, emulsions, creams, ointments, suppositories, inhalable forms, transdermal forms, and the like. The quinine formulation may be a dosage form administered via oral, buccal, injectable, or transdermal administration.

By “oral dosage form” is meant to include a dosage form prescribed or intended for oral administration. An oral dosage form may or may not comprise a plurality of subunits such as, for example, microcapsules or microtablets, packaged for administration in a single dose. The oral dosage form can be in solid or liquid form.

By an “effective” amount or a “therapeutically effective amount” of an active agent is meant a sufficient amount of the active agent to produce a therapeutic effect in the patient. The amount that is “effective” will vary from subject to subject, depending on the age and general condition of the individual, the particular active agent, and the like. Thus, it is not always possible to specify an exact “effective amount.” However, an appropriate “effective” amount in any individual case may be determined by one of ordinary skill in the art using routine experimentation.

“Efficacy” means the ability of an active agent administered to a patient to produce a therapeutic effect in the patient.

A “patient” means a human or non-human animal in need of medical treatment. Medical treatment can include treatment of an existing condition, such as a disease or disorder, prophylactic or preventative treatment, or diagnostic treatment. In some embodiments the patient is a human patient. A “caretaker” includes a worker in the health care field, physicians, pharmacists, physician's assistants, nurses, aides, caretakers (which can include family members or guardians), emergency medical workers, and the like.

The terms “treating” and “treatment” mean the reduction in severity or frequency of symptoms, elimination of symptoms or underlying cause, prevention of the occurrence of symptoms or their underlying cause, and improvement or remediation of damage.

A “product” or “pharmaceutical product” means a dosage form of an active agent and optionally packaging.

“Safety” means the incidence or severity of adverse events associated with administration of an active agent, including adverse effects associated with patient-related factors (e.g., age, gender, ethnicity, race, target illness, abnormalities of renal or hepatic function, co-morbid illnesses, genetic characteristics such as metabolic status, or environment) and active agent-related factors (e.g., dose, plasma level, duration of exposure, or concomitant medication).

By “releasable form” is meant to include immediate-release, controlled-release, and extended-release forms. Certain release forms can be characterized by their dissolution profile. Dissolution profile as used herein, means a plot of the amount of active ingredient released as a function of time. The dissolution profile may be measured utilizing the Drug Release Test <724>, which incorporates standard test USP 28 (Test <711>) or by other test methods or conditions. A profile is characterized by the test conditions selected. Thus the dissolution profile can be generated at a preselected apparatus type, shaft speed, temperature, volume, and pH of the dissolution media.

A first dissolution profile can be measured at a pH level approximating that of the stomach. A second dissolution profile can be measured at a pH level approximating that of one point in the intestine or several pH levels approximating multiple points in the intestine.

A highly acidic pH may simulate the stomach and a less acidic to basic pH may simulate the intestine. By the term “highly acidic pH”: it is meant a pH of about 1 to about 4. By the term “less acidic to basic pH” is meant a pH of greater than about 4 to about 7.5, specifically about 6 to about 7.5. A pH of about 1.2 can be used to simulate the pH of the stomach. A pH of about 6 to about 7.5, specifically about 6.8, can be used to simulate the pH of the intestine.

“Pharmacokinetic parameters” describe the in vivo characteristics of an active agent (or surrogate marker for the active agent) over time, such as plasma concentration (C), Cₘₐₓ, Cₙ, C₂₄, Tₘₐₓ, and AUC. “Cₘₐₓ” is the measured concentration of the active agent in the plasma at the point of maximum concentration. “Cₙ” is the measured concentration of an active agent in the plasma at about n hours after administration. “C₂₄” is the measured concentration of an active agent in the plasma at about 24 hours after administration. The term “Tₘₐₓ” refers to the time at which the measured concentration of an active agent in the plasma is the highest after administration of the active agent. “AUC” is the area under the curve of a graph of the measured concentration of an active agent (typically plasma concentration) vs. time, measured from one time point to another time point. For example AUC₀₋ₜ is the area under the curve of plasma concentration versus time from time 0 to time t. The AUC_{0-∞} (AUC_{∞}) or AUC_{0-INF} (AUC_{inf}) is the calculated area under the curve of plasma concentration versus time from time 0 to time infinity.

By “immediate-release”, it is meant a conventional or non-modified release in which greater than or equal to about 75% of the active agent is released within two hours of administration, specifically within one hour of administration. Alternatively, an “immediate-release” formulation contains substantially no added release retarding agents.

By “controlled-release” it is meant a dosage form in which the release of the active agent is controlled or modified over a period of time. Controlled can mean, for example, extended- or delayed-release at a particular time. Alternatively, controlled can mean that the release of the active agent is extended for longer than it would be in an immediate-release dosage form, i.e., at least over several hours.

“Sustained-release” or “extended-release” include the release of the active agent at such a rate that blood (e.g., plasma) levels are maintained within a therapeutic range for at least about 8 hours, specifically at least about 12 hours, and more specifically at least about 24 hours after administration at steady-state. The term steady-state means that a plasma level for a given active agent has been achieved and which is maintained with subsequent doses of the drug at a level which is at or above the minimum effective therapeutic level for a given active agent.

By “delayed-release”, it is meant that there is a time-delay before significant plasma levels of the active agent are achieved. A delayed-release formulation of the active agent can avoid an initial burst of the active agent, or can be formulated so that release of the active agent in the stomach is avoided and absorption occurrs in the small intestine.

An extended-release form is a form suitable for providing controlled-release of quinine over a sustained period of time (e.g., 8 hours, 12 hours, 24 hours). Extended-release dosage forms of quinine may release the active agent at a rate independent of pH, for example, about pH 1.2 to about 7.5. Alternatively, extended-release dosage forms may release quinine at a rate dependent upon pH, for example, a lower rate of release at pH 1.2 and a higher rate of release at pH 7.5. Specifically, the extended-release form avoids dose dumping upon oral administration. The extended-release oral dosage form can be formulated to provide for an increased duration of quinine action allowing once-daily or twice-daily dosing.

There are several approaches to preparing an extended-release quinine dosage formulation. Exemplary forms include polymeric matrices containing quinine, coated tablets, coated particles, osmotic pump, depot forms, and the like. Each will be discussed herein below.

Generally, an extended-release dosage form comprises a release-retarding material. The release-retarding material can be, for example, in the form of a matrix or a coating. The quinine in extended-release form may be, for example, a particle of quinine that is combined with a release-retarding material. The release-retarding material is a material that permits release of the active agent at a sustained rate in an aqueous medium. The release-retarding material can be selectively chosen so as to achieve, in combination with the other stated properties, a desired in vitro release rate.

Release-retarding materials include, for example acrylic polymers, alkylcelluloses, shellac, zein, hydrogenated vegetable oil, hydrogenated castor oil, polyvinylpyrrolidine, vinyl acetate copolymers, polyethylene oxide, and a combination comprising at least one of the foregoing materials. The extended-release oral dosage form can contain between about 1 wt % and about 80 wt % of the release-retarding material based on the total weight of the oral dosage form.

Suitable acrylic polymers that can be used as release-retarding materials include, for example, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, glycidyl methacrylate copolymers, and a combination comprising at least one of the foregoing polymers. The acrylic polymer may comprise methacrylate copolymers described in NF XXIV as fully polymerized copolymers of acrylic and methacrylic acid esters with a low content of quaternary ammonium groups.

Suitable alkylcelluloses include, for example, methyl cellulose, ethylcellulose, and the like. Those skilled in the art will appreciate that other cellulosic polymers, including other alkyl cellulosic polymers, can be substituted for part or all of the ethylcellulose.

Other suitable release-retarding materials include neutral or synthetic waxes, fatty alcohols (such as lauryl, myristyl, stearyl, cetyl or specifically cetostearyl alcohol), fatty acids, including fatty acid esters, fatty acid glycerides (mono-, di-, and tri-glycerides), hydrogenated fats, hydrocarbons, normal waxes, stearic acid, stearyl alcohol, hydrophobic and hydrophilic materials having hydrocarbon backbones, and a combination comprising at least one of the foregoing materials. Suitable waxes include beeswax, glycowax, castor wax, carnauba wax and wax-like substances, e.g., material normally solid at room temperature and having a melting point of from about 30° C. to about 100° C., and a combination comprising at least one of the foregoing waxes.

In other embodiments, the release-retarding material may comprise digestible, long chain (e.g., C₈-C₅₀, specifically C₁₂-C₄₀), substituted or unsubstituted hydrocarbons, such as fatty acids, fatty alcohols, glyceryl esters of fatty acids, mineral and vegetable oils, waxes, and a combination comprising at least one of the foregoing materials. Hydrocarbons having a melting point of between about 25° C. and about 90° C. may be used. Specifically, long chain hydrocarbon materials, fatty (aliphatic) alcohols can be used. The oral dosage form can contain up to about 60 wt % of a digestible, long chain hydrocarbon, based on the total weight of the oral dosage form.

Further, the extended-release matrix can contain up to about 60 wt % of a polyalkylene glycol.

Alternatively, the release-retarding material may comprise polylactic acid, polyglycolic acid, or a co-polymer of lactic and glycolic acid.

Alternatively, the release-retarding material can include, for example, crosslinked sodium carboxymethylcellulose, crosslinked hydroxypropylcellulose, high molecular weight hydroxypropylmethylcellulose, carboxymethyl starch, potassium methacrylate/divinylbenzene copolymer, polymethylmethacrylate, crosslinked polyvinylpyrrolidone, high molecular weight polyvinylalcohols, methylcellulose, carboxymethylcellulose, low molecular weight hydroxypropylmethylcellulose, low molecular weight polyvinylalcohols, polyethylene glycols, non-crosslinked polyvinylpyrrolidone, medium viscosity hydroxypropylmethylcellulose, medium viscosity polyvinylalcohols, combinations thereof and the like.

Release-modifying agents, which affect the release properties of the release-retarding material, can optionally be used. The release-modifying agent can, for example, function as a pore-former. The pore former can be organic or inorganic, and include materials that can be dissolved, extracted or leached from the material in the environment of use. The pore-former can comprise one or more hydrophilic polymers, such as hydroxypropylmethylcellulose, hydroxypropylcellulose, polycarbonates comprised of linear polyesters of carbonic acid in which carbonate groups reoccur in the polymer chain, and a combination comprising at least one of the foregoing release-modifying agents. Alternatively, the pore-former may be a small molecule such as lactose, or metal stearates, and a combination comprising at least one of the foregoing release-modifying agents.

The release-retarding material can also optionally include other additives such as an erosion-promoting agent (e.g., starch and gums); and/or a semi-permeable polymer. In addition to the above ingredients, an extended-release dosage form may also contain suitable quantities of other materials, e.g., diluents, lubricants, binders, granulating aids, colorants, flavorants and glidants that are conventional in the pharmaceutical art. The release-retarding material can also include an exit means comprising a passageway, orifice, or the like. The passageway can have any shape, such as round, triangular, square, elliptical, irregular, etc.

The extended-release dosage form comprising quinine or a salt thereof and a release-retarding material may be prepared by a suitable technique for preparing active agents as described in detail below. The quinine or a salt thereof and release-retarding material may, for example, be prepared by wet granulation techniques, melt extrusion techniques, etc. To obtain an extended-release dosage form, it may be advantageous to incorporate an additional hydrophobic material.

The quinine or salt thereof in extended-release form can include a plurality of substrates (particles such as microparticles) comprising the active agent, which substrates are coated with an extended-release coating comprising a release-retarding material. The extended-release preparations may thus be made in conjunction with a multiparticulate system, such as beads, ion-exchange resin beads, spheroids, microspheres, seeds, pellets, granules, and other multiparticulate systems in order to obtain a desired extended-release of the quinine or salt thereof. The multiparticulate system can be presented in a capsule or other suitable unit dosage form.

In certain cases, more than one multiparticulate system can be used, each exhibiting different characteristics, such as pH dependence of release, time for release in various media (e.g., acid, base, simulated intestinal fluid), release in vivo, size, and composition.

In some cases, a spheronizing agent, together with the quinine or salt thereof can be spheronized to form spheroids. Microcrystalline cellulose and hydrous lactose impalpable are examples of such agents. Additionally (or alternatively), the spheroids can contain a water insoluble polymer, specifically an acrylic polymer, an acrylic copolymer, such as a methacrylic acid-ethyl acrylate copolymer, or ethyl cellulose. In this formulation, the extended-release coating will generally include a water insoluble material such as a wax, either alone or in admixture with a fatty alcohol, or shellac or zein.

Spheroids or beads, coated with quinine or a salt thereof can be prepared, for example, by dissolving or dispersing the active agent in a solvent and then spraying the solution onto a substrate, for example, sugar spheres NF, 18/20 mesh, using a Wurster insert. Optionally, additional ingredients are also added prior to coating the beads in order to assist the quinine or salt thereof binding to the substrates, and/or to color the resulting beads, etc. The resulting substrate-active agent may optionally be overcoated with a barrier material, to separate the therapeutically active agent from the next coat of material, e.g., release-retarding material. For example, the barrier material is a material comprising hydroxypropylmethylcellulose. However, film-formers known in the art may be used.

To obtain a extended-release of quinine or salt thereof in a manner sufficient to provide a therapeutic effect for the sustained durations, the substrate comprising the active agent can be coated with an amount of release-retarding material sufficient to obtain a weight gain level from about 2 wt % to about 30 wt %, specifically about 5 wt % to about 25 wt %, and more specifically about 7 wt % to about 20 wt %, although the coat can be greater or lesser depending upon the physical properties of the active agent utilized and the desired release rate, among other things. Moreover, there can be more than one release-retarding material used in the coat, as well as various other pharmaceutical excipients.

The release-retarding material may thus be in the form of a film coating comprising a dispersion of a hydrophobic polymer. Solvents used for application of the release-retarding coating include pharmaceutically acceptable solvents, such as water, methanol, ethanol, methylene chloride, and a combination comprising at least one of the foregoing solvents.

In addition, the extended-release profile of quinine or salt thereof (either in vivo or in vitro) can be altered, for example, by using more than one release-retarding material, varying the thickness of the release-retarding material, changing the particular release-retarding material used, altering the relative amounts of release-retarding material, altering the manner in which the plasticizer is added (e.g., when the extended-release coating is derived from an aqueous dispersion of hydrophobic polymer), by varying the amount of plasticizer relative to retardant material, by the inclusion of additional ingredients or excipients, by altering the method of manufacture, etc.

The extended-release formulations preferably slowly release quinine or salt thereof, e.g., when ingested and exposed to gastric fluids, and then to intestinal fluids. The extended-release profile of the formulations can be altered, for example, by varying the amount of retardant, e.g., hydrophobic material, by varying the amount of plasticizer relative to hydrophobic material, by the inclusion of additional ingredients or excipients, by altering the method of manufacture, etc.

Exemplary forms containing a release-retarding material coating can comprise quinine blended with a water soluble polymer that is a film forming polymer. Useful water soluble film forming polymers are polymers that have an apparent viscosity of 1 to 100 mPa·s when dissolved in a 2% aqueous solution at 20° C. solution. For example, the water soluble film forming polymers can be selected from the group comprising alkylcelluloses such as methylcellulose, hydroxyalkylcelluloses such as hydroxymethylcellulose, hydroxyethylcellulose, hydroxypropylcellulose and hydroxybutylcellulose, hydroxyalkyl alkylcelluloses such as hydroxyethyl methylcellulose and hydroxypropyl methylcellulose, carboxyalkylcelluloses such as carboxymethylcellulose, alkali metal salts of carboxyalkylcelluloses such as sodium carboxymethylcellulose, carboxyalkyl alkylcelluloses such as carboxymethyl ethylcellulose, carboxyalkylcellulose esters, starches, pectines such as sodium carboxymethylamylopectine, chitine derivates such as chitosan, polysaccharides such as alginic acid, alkali metal and ammonium salts thereof, carrageenans, galactomannans, traganth, agar-agar, gum arabicum, guar gum and xanthan gum, polyacrylic acids and the salts thereof, polymethacrylic acids and the salts thereof, methacrylate copolymers, polyvinylalcohol, polyvinylpyrrolidone, copolymers of polyvinylpyrrolidone with vinyl acetate, polyalkylene oxides such as polyethylene oxide and polypropylene oxide and copolymers of ethylene oxide and propylene oxide. Other pharmaceutically acceptable polymers that exhibit similar as defined above physico-chemical properties as defined above are equally suitable.

Specific water soluble film forming polymers are for example hydroxypropyl methylcellulose, polymethacrylate, hydroxypropylcellulose, or a polyvidone; more specifically hydroxypropyl methylcelluloses (HPMCs). HPMCs contain sufficient hydroxypropyl and methoxy groups to render it water-soluble. HPMC having a methoxy degree of substitution from about 0.8 to about 2.5 and a hydroxypropyl molar substitution from about 0.05 to about 3.0 are generally water-soluble. Methoxy degree of substitution refers to the average number of methyl ether groups present per anhydroglucose unit of the cellulose molecule. Hydroxypropyl molar substitution refers to the average number of moles of propylene oxide which have reacted with each anhydroglucose unit of the cellulose molecule. Suitable HPMC include those having a viscosity from about 1 to about 100 mPa·s, specifically about 3 to about 15 mPa·s, and more specifically about 5 mPa·s.

The weight-by-weight ratio of drug:water soluble film forming polymer is in the range of about 17:1 to about 1:5, specifically about 10:1 to about 1:3, and more specifically about 7:1 to about 1:2.

The particles generally comprise (a) a central, rounded or spherical core, (b) a layer or a coating film of a water soluble film forming polymer and quinine or a salt therof, (c) optionally a barrier polymer layer and (d) a release retarding material coating. The core can have a diameter of about 250 to about 2000 micrometers, specifically about 600 to about 1500 micrometers, and yet more specifically about 750 to about 1000 micrometers.

Materials suitable for use as the cores of the particles include pharmaceutically acceptable materials that have appropriate dimensions and firmness. Examples of such materials are polymers e.g. plastic resins; inorganic substances, e.g. silica, glass, hydroxyapatite, salts (sodium or potassium chloride, calcium or magnesium carbonate) and the like; organic substances, e.g. activated carbon, acids (citric, fumaric, tartaric, ascorbic and the like acids), and saccharides and derivatives thereof. Particularly suitable materials are saccharides such as sugars, oligosaccharides, polysaccharides and their derivatives, for example, glucose, rhamnose, galactose, lactose, sucrose, mannitol, sorbitol, dextrin, maltodextrin, cellulose, microcrystalline cellulose, sodium carboxymethyl cellulose, starches (maize, rice, potato, wheat, tapioca) and the like saccharides.

The combination of the water soluble film forming polymer and quinine can be coated on the core as a layer to form a coated core.

In another embodiment, the cores themselves can contain quinine. The cores containing quinine can be granules or spheroids (spherical granules) prepared according to art-known methods of granulation and spheronization.

The particles can be filled in hard-gelatin capsules such that a therapeutically effective amount of the active ingredient is available per dosage form. An desired pharmacokinetic profile (fast onset, level peak and trough values) can be obtained when about 60 to about 90 weight % of the quinine based on the total amount of quinine in the dosage form, specifically about 70 to about 80 weight % of the quinine is comprised within the controlled-release particles and about 10 to about 40 weight %, specifically about 20 to about 30 weight % of the quinine based on the total amount of quinine in the dosage form, is in an immediate-release form.

In order to achieve the desired pharmacokinetic profile, the dosage forms may be filled with particles that release quinine at different rates, a kind that releases quinine slowly, and a kind that releases quinine more rapidly, in particular one kind that releases the active ingredient immediately, e.g. particles as described that lack the release retarding material coating.

The different particles may be filled consecutively in the capsules, or they may be premixed and the thus obtained premix may be filled into the capsules (taking into account possible segregation).

Alternatively, the controlled-release particles may further comprise a top-coat of a water-soluble polymer as described hereinbefore and quinine which is released practically immediately upon ingestion and thus ensures a rapid onset of action.

In another embodiment, a capsule is filled with controlled-release particles as described above (about 60 to about 90 weight %, specifically about 70 to about 80 weight % based on the total weight of quinine in the dosage form) together with one or more minitablets which comprise the remaining amount of quinine.

The quinine formulations can be coated with a material to delay release of the quinine until the formulation is exposed to the intestinal tract. These formulations include enteric coated formulations, which are forms coated with a composition that is non-toxic and includes a pharmaceutically acceptable enteric polymer which is predominantly soluble in the intestinal fluid, but substantially insoluble in the gastric juices. An enteric coating is a coating that prevents release of the active agent until the dosage form reaches the small intestine. Enteric coated dosage forms comprise quinine or a salt thereof coated with an enteric polymer. Examples include polyvinyl acetate phthalate (PVAP), hydroxypropylmethyl-cellulose acetate succinate (HPMCAS), cellulose acetate phthalate (CAP), methacrylic acid copolymer, hydroxy propyl methylcellulose succinate, cellulose acetate succinate, cellulose acetate hexahydrophthalate, hydroxypropyl methylcellulose hexahydrophthalate, hydroxypropyl methylcellulose phthalate (HPMCP), cellulose propionate phthalate, cellulose acetate maleate, cellulose acetate trimellitate, cellulose acetate butyrate, cellulose acetate propionate, methacrylic acid/methacrylate polymer (acid number 300 to 330 and also known as EUDRAGIT L), which is an anionic copolymer based on methacrylate and available as a powder (also known as methacrylic acid copolymer, type A NF, methacrylic acid-methyl methacrylate copolymer, ethyl methacrylate-methylmethacrylate-chlorotrimethylammonium ethyl methacrylate copolymer, and the like, and a combination comprising at least one of the foregoing enteric polymers. Other examples include natural resins, such as shellac, SANDARAC, copal collophorium, and a combination comprising at least one of the foregoing polymers. Yet other examples of enteric polymers include synthetic resin bearing carboxyl groups. The methacrylic acid: acrylic acid ethyl ester 1:1 copolymer solid substance of the acrylic dispersion sold under the trade designation “EUDRAGIT L-100-55” may be suitable.

The extended-release quinine formulations can be prepared to include an immediate-release portion. An exemplary form may provide at least a part of the dose with an extended-release of quinine and another part of the formulation with rapid or immediate-release. The immediate- and extended-release of quinine can be achieved according to different principles, such as by single dose layered pellets or tablets, by multiple dose layered pellets or tablets, or by two or more different fractions of single or multiple dose layered pellets or tablets, optionally in combination with pellets or tablets having instant release. Multiple dose layered pellets may be filled into a capsule or together with tablet excipients compressed into a multiple unit tablet. Alternatively, a multiple dose layered tablet may be prepared.

Pellets or tablets may comprise a core material, optionally layered on a seed/sphere, the core material comprising quinine together with a water swellable substance; an optional intermediate layer surrounding the core; and an outer coating layer containing quinine in an immediate-release form. Alternatively, the layered pellets or tablets may comprise a core material comprising quinine; a surrounding layer comprising a water swellable substance; an outer coating layer containing quinine in an immediate-release form; and optional intermediate layers for ease of processing or improved dosage form stability.

In another embodiment, part of the quinine is present in an immediate-release form, for example, as particles lacking a release-retarding material coating, or as immediate-release minitablets, or as a topcoat on the extended-release formulation.

The quinine or pharmaceutically acceptable salt thereof can also be formulated with OROS technology (Alza Corporation, Mountain View, Calif.) also know as an “osmotic pump”. Such dosage forms have a fluid-permeable (semipermeable) membrane wall, an osmotically active expandable driving member (the osmotic push layer), and a density element for delivering the active agent. In an osmotic pump dosage form, quinine may be dispensed through an exit means comprising a passageway, orifice, or the like, by the action of the osmotically active driving member. The active agent of the osmotic pump dosage form may be formulated as a thermo-responsive formulation in which the quinine is dispersed in a thermo-responsive composition. Alternatively, the osmotic pump dosage form may contain a thermo-responsive element comprising a thermo-responsive composition at the interface of the osmotic push layer and the quinine composition.

The term “thermo-responsive” as used herein includes thermoplastic compositions capable of softening, or becoming dispensable in response to heat and hardening again when cooled. The term also includes thermotropic compositions capable of undergoing changes in response to the application of energy in a gradient manner. These compositions are temperature sensitive in their response to the application or withdrawal of energy. Thermo-responsive compositions typically possess the physiochemical property of exhibiting solid, or solid-like properties at temperatures up to about 32° C., and become fluid, semisolid, or viscous when at temperatures above about 32° C., usually in about 32° C. to about 40° C. Thermo-responsive compositions, including thermo-responsive carriers, have the property of melting, dissolving, undergoing dissolution, softening, or liquefying and thereby forming a dispensable composition at the elevated temperatures. The thermo-responsive carrier can be lipophilic, hydrophilic, or hydrophobic. Another property of a thermo-responsive carrier is its ability to maintain the stability of the agent contained therein during storage and during delivery of the agent. A thermo-responsive composition can be easily excreted, metabolized, or assimilated, upon being dispensed into a biological environment.

The osmotic pump dosage form comprises a semipermeable membrane. The capsule or other dispenser of the osmotic pump dosage form can be provided with an outer wall comprising the selectively semipermeable material. A selectively permeable material is one that does not adversely affect a host or animal, is permeable to the passage of an external aqueous fluid, such as water or biological fluids, while remaining essentially impermeable to the passage of the active agent, and maintains its integrity in the presence of a thermotropic thermo-responsive composition, that is it does not melt or erode in its presence. The selectively semipermeable material forming the outer wall is substantially insoluble in body fluids, nontoxic, and non-erodible.

Representative materials for forming the selectively semipermeable wall include semipermeable homopolymers, semipermeable copolymers, and the like. Suitable materials include, for example, cellulose esters, cellulose monoesters, cellulose diesters, cellulose triesters, cellulose ethers, cellulose ester-ethers, and a combination comprising at least one of the foregoing materials. These cellulosic polymers have a degree of substitution, D.S., on their anhydroglucose unit from greater than 0 up to 3 inclusive. By degree of substitution is meant the average number of hydroxyl groups originally present on the anhydroglucose unit that are replaced by a substituting group, or converted into another group. The anhydroglucose unit can be partially or completely substituted with groups such as acyl, alkanoyl, aroyl, alkyl, alkenyl, alkoxy, halogen, carboalkyl, alkylcarbamate, alkylcarbonate, alkylsulfonate, alkylsulfamate, and like semipermeable polymer forming groups.

Other selectively semipermeable materials include, for example, cellulose acylate, cellulose diacylate, cellulose triacylate, cellulose acetate, cellulose diacetate, cellulose triacetate, mono-, di- and tri-cellulose alkanylates, mono-, di- and tri-alkenylates, mono-, di- and tri-aroylates, and the like, and a combination comprising at least one of the foregoing materials. Exemplary polymers including cellulose acetate having a D.S. of 1.8 to 2.3 and an acetyl content of about 32 to about 39.9%; cellulose diacetate having a D.S. of 1 to 2 and an acetyl content of about 21 to about 35%; cellulose triacetate having a D.S of 2 to 3 and an acetyl content of about 34 to about 44.8%, and the like. More specific cellulosic polymers include cellulose propionate having a D.S. of 1.8 and a propionyl content of about 38.5%; cellulose acetate propionate having an acetyl content of about 1.5 to about 7% and an propionyl content of about 39 to about 42%; cellulose acetate propionate having an acetyl content of about 2.5 to about 3%, an average propionyl content of about 39.2 to about 45% and a hydroxyl content of about 2.8 to about 5.4%; cellulose acetate butyrate having a D.S. of 1.8, an acetyl content of about 13 to about 15%, and a butyryl content of about 34 to about 39%; cellulose acetate butyrate having an acetyl content of about 2 to about 29.5%, a butyryl content of about 17 to about 53%, and a hydroxyl content of about 0.5 to about 4.7%; cellulose triacylates having a D.S. of 2.9 to 3 such as cellulose trivalerate, cellulose trilaurate, cellulose tripalmitate, cellulose trioctanoate, and cellulose tripropionate; cellulose diesters having a D.S. of 2.2 to 2.6 such as cellulose disuccinate, cellulose dipalmitate, cellulose dioctanoate, cellulose dicarpylate and the like; mixed cellulose esters such as cellulose acetate valerate, cellulose acetate succinate, cellulose propionate succinate, cellulose acetate octanoate, cellulose valerate palmitate, cellulose acetate heptanoate, and the like, and a combination comprising at least one of the foregoing polymers.

Additional selectively semipermeable polymers include, for example, acetaldehyde dimethyl cellulose acetate, cellulose acetate ethylcarbamate, cellulose acetate methylcarbamate, cellulose dimethylaminoacetate, semi-permeable polyamides, semipermeable polyurethanes, semi-permeable polysulfanes, semipermeable sulfonated polystyrenes, cross-linked, selectively semipermeable polymers formed by the coprecipitation of a polyanion and a polycation, selectively semipermeable silicon rubbers, semipermeable polystyrene derivates, semipermeable poly(sodium styrenesulfonate), semipermeable poly(vinylbenzyltrimethyl) ammonium chloride polymers, and a combination comprising at least one of the foregoing polymers.

The osmotically expandable driving member, or osmotic push layer, of the osmotic pump dosage form is swellable and expandable inner layer. The materials used for forming the osmotic push layer, are neat polymeric materials, and/or polymeric materials blended with osmotic agents that interact with water or a biological fluid, absorb the fluid, and swell or expand to an equilibrium state. The polymer should exhibit the ability to retain a significant fraction of imbibed fluid in the polymer molecular structure. Such polymers may be, for example, gel polymers that can swell or expand to a very high degree, usually exhibiting about a 2 to 50-fold volume increase. Swellable, hydrophilic polymers, also known as osmopolymers, can be non-cross-linked or lightly cross-linked. The cross-links can be covalent or ionic bonds with the polymer possessing the ability to swell but not dissolve in the presence of fluid. The polymer can be of plant, animal or synthetic origin. Polymeric materials useful for the present purpose include poly(hydroxyalkyl methacrylate) having a molecular weight of about 5,000 to about 5,000,000, poly(vinylpyrrolidone) having a molecular weight of about 10,000 to about 360,000, anionic and cationic hydrogels, poly(electrolyte) complexes, poly(vinyl alcohol) having a low acetate residual, a swellable mixture of agar and carboxymethyl cellulose, a swellable composition comprising methyl cellulose mixed with a sparingly crosslinked agar, a water-swellable copolymer produced by a dispersion of finely divided copolymer of maleic anhydride with styrene, ethylene, propylene, or isobutylene, water swellable polymer of N-vinyl lactams, and the like, and a combination comprising at least one of the foregoing polymers. Other gellable, fluid imbibing and retaining polymers useful for forming the osmotic push layer include pectin having a molecular weight ranging of about 30,000 to about 300,000, polysaccharides such as agar, acacia, karaya, tragacanth, algins and guar, acidic carboxy polymer and its salt derivatives, polyacrylamides, water-swellable indene maleic anhydride polymers; polyacrylic acid having a molecular weight of about 80,000 to about 200,000; POLYOX, polyethylene oxide polymers having a molecular weight of about 100,000 to about 5,000,000, and greater, starch graft copolymers, polyanions and polycations exchange polymers, starch-polyacrylonitrile copolymers, acrylate polymers with water absorbability of about 400 times its original weight, diesters of polyglucan, a mixture of cross-linked polyvinyl alcohol and poly(N-vinyl-2-pyrrolidone), zein available as prolamine, poly(ethylene glycol) having a molecular weight of about 4,000 to about 100,000, and the like, and a combination comprising at least one of the foregoing polymers.

The osmotically expandable driving layer of the osmotic pump dosage form may further contain an osmotically effective compound (osmagent) that can be used neat or blended homogeneously or heterogeneously with the swellable polymer, to form the osmotically expandable driving layer. Such osmagents include osmotically effective solutes that are soluble in fluid imbibed into the swellable polymer, and exhibit an osmotic pressure gradient across the semipermeable wall against an exterior fluid. Suitable osmagents include, for example, solid compounds such as magnesium sulfate, magnesium chloride, sodium chloride, lithium chloride, potassium sulfate, sodium sulfate, mannitol, urea, sorbitol, inositol, sucrose, glucose, and the like, and a combination comprising at least one of the foregoing osmagents. The osmotic pressure in atmospheres, atm, of the osmagents may be greater than about zero atm, and generally about zero atm to about 500 atm, or higher.

The swellable, expandable polymer of the osmotically expandable driving layer, in addition to providing a driving source for delivering the active agent from the dosage form, may also function as a supporting matrix for an osmotically effective compound. The osmotic compound can be homogeneously or heterogeneously blended with the polymer to yield the desired expandable wall or expandable pocket. The composition in a presently preferred embodiment comprises (a) a polymer and an osmotic compound, or (b) a solid osmotic compound. Generally, a composition will comprise about 20 wt % to about 90 wt % of polymer and about 10 wt % to about 80 wt % of osmotic compound, with a presently preferred composition comprising about 35 wt % to about 75 wt % of polymer and about 25 wt % to about 65 wt % of osmotic compound, based on the total weight of the composition.

The quinine of the osmotic pump dosage form may be formulated as a thermo-responsive formulation in which the quinine is dispersed in a thermo-responsive composition. Alternatively, the osmotic pump dosage form may contain a thermo-responsive element comprising a thermo-responsive composition at the interface of the osmotic push layer and the quinine composition. Representative thermo-responsive compositions and their melting points are as follows: Cocoa butter (32° C.-34° C.), cocoa butter plus 2% beeswax (35° C.-37° C.), propylene glycol monostearate and distearate (32° C.-35° C.), hydrogenated oils such as hydrogenated vegetable oil (36° C.-37.5° C.), 80% hydrogenated vegetable oil and 20% sorbitan monopalmitate (39° C.-39.5° C.), 80% hydrogenated vegetable oil and 20% polysorbate 60, (36° C.-37° C.), 77.5% hydrogenated vegetable oil, 20% sorbitan trioleate, 2.5% beeswax and 5.0% distilled water, (37° C.-38° C.), mono-, di-, and triglycerides of acids having from 8-22 carbon atoms including saturated and unsaturated acids such as palmitic, stearic, oleic, lineolic, linolenic and archidonic; triglycerides of saturated fatty acids with mono- and diglycerides (34° C.-35.5° C.), propylene glycol mono- and distearates 3(33° C.-34° C.), partially hydrogenated cottonseed oil (35° C.-39° C.), a block polymer of polyoxy-alkylene and propylene glycol; block polymers comprising 1,2-butylene oxide to which is added ethylene oxide; block copolymers of propylene oxide and ethylene oxide, hardened fatty alcohols and fats (33° C.-36° C.), hexadienol and hydrous lanolin triethanolamine glyceryl monostearate (38° C.), eutectic mixtures of mono-, di-, and triglycerides (35° C.-39° C.), WITEPSOL#15, triglyceride of saturated vegetable fatty acid with monoglycerides (33.5° C.-35.5° C.), WITEPSOL H32 free of hydroxyl groups (31° C.-33° C.), WITEPSOL W25 having a saponification value of 225-240 and a melting point of (33.5° C.-35.5° C.), WITEPSOL E75 having a saponification value of 220-230 and a melting point of (37° C.-39° C.), a polyalkylene glycol such as polyethylene glycol 1000, a linear polymer of ethylene oxide (38° C.-41° C.), polyethylene glycol 1500 (38° C.-41° C.), polyethylene glycol monostearate (39° C.-42.5° C.), 33% polyethylene glycol 1500, 47% polyethylene glycol 6000 and 20% distilled water (39° C.-41° C.), 30% polyethylene glycol 1500, 40% polyethylene glycol 4000 and 30% polyethylene glycol 400, (33° C.-38° C.), mixture of mono-, di-, and triglycerides of saturated fatty acids having 11 to 17 carbon atoms, (33° C.-35° C.), and the like. The thermo-responsive compositions, including thermo-responsive carriers are useful for storing the active agent in a solid composition at a temperature of about 20° C. to about 33° C., maintaining an immiscible boundary at the swelling composition interface, and for dispensing the agent in a flowable composition at a temperature greater than about 33° C. and specifically between about about 33° C. and about 40° C.

The amount of quinine present in the osmotic pump dosage form is about 10 mg to about 2 g or more. The osmotic dosage form may be formulated for once daily or less frequent administration.

The quinine of the osmotic pump dosage form may be formulated by a number of techniques known in the art for formulating solid and liquid oral dosage forms. The quinine of the osmotic pump dosage form may be formulated by wet granulation. In an exemplary wet granulation method, the quinine and the ingredients comprising the quinine layer are blended using an organic solvent, such as isopropyl alcohol-ethylene dichloride 80:20 v:v (volume:volume) as the granulation fluid. Other granulating fluid such as denatured alcohol 100% may be used for this purpose. The ingredients forming the quinine layer are individually passed through a screen such as a 40-mesh screen and then thoroughly blended in a mixer. Next, other ingredients comprising the active agent layer are dissolved in a portion of the granulation fluid, such as the cosolvent described above. Then the latter prepared wet blend is slowly added to the active agent blend with continual mixing in the blender. The granulating fluid is added until a wet blend is produced, which wet mass then is forced through a screen such as a 20-mesh screen onto oven trays. The blend is dried for about 18 to about 24 hours at about 30° C. to about 50° C. The dry granules are sized then with a screen such as a 20-mesh screen. Next, a lubricant is passed through a screen such as an 80-mesh screen and added to the dry screen granule blend. The granulation is put into milling jars and mixed on a jar mill for about 1 to about 15 minutes. The push layer may also be made by the same wet granulation techniques. The compositions are pressed into their individual layers in a KILIAN press-layer press.

Another manufacturing process that can be used for providing the quinine layer and osmotically expandable driving layer comprises blending the powered ingredients for each layer independently in a fluid bed granulator. After the powered ingredients are dry blended in the granulator, a granulating fluid, for example, poly(vinyl-pyrrolidone) in water, or in denatured alcohol, or in 95:5 ethyl alcohol/water, or in blends of ethanol and water is sprayed onto the powders. Optionally, the ingredients can be dissolved or suspended in the granulating fluid. The coated powders are then dried in a granulator. This process granulates the ingredients present therein while adding the granulating fluid. After the granules are dried, a lubricant such as stearic acid or magnesium stearate is added to the granulator. The granules for each separate layer are pressed then in the manner described above.

The quinine formulation and osmotic push layer of the osmotic dosage form may also be manufactured by mixing quinine with composition forming ingredients and pressing the composition into a solid lamina possessing dimensions that correspond to the internal dimensions of the compartment. In another manufacture, quinine and other quinine composition-forming ingredients and a solvent are mixed into a solid, or a semisolid, by methods such as ballmilling, calendaring, stirring or rollmilling, and then pressed into a preselected layer forming shape. Next, a layer of a composition comprising an osmopolymer and an optional osmagent are placed in contact with the layer comprising the quinine. The layering of the first layer comprising the quinine and the second layer comprising the osmopolymer and optional osmagent composition can be accomplished by using a conventional layer press technique. The semipermeable wall can be applied by molding, spraying or dipping the pressed bilayer's shapes into wall forming materials. An air suspension coating procedure which includes suspending and tumbling the two layers in current of air until the wall forming composition surrounds the layers is also used to form the semi-permeable wall of the osmotic dosage forms.

The dispenser of the osmotic pump dosage form may be in the form of a capsule. The capsule may comprise an osmotic hard capsule and/or an osmotic soft capsule. The osmotic hard capsule may be composed of two parts, a cap and a body, which are fitted together after the larger body is filled with the active agent. The osmotic hard capsule may be fitted together by slipping or telescoping the cap section over the body section, thus completely surrounding and encapsulating the active agent. Hard capsules may be made by techniques known in the art.

The soft capsule of the osmotic pump dosage form may be a one-piece osmotic soft capsule. Generally, the osmotic soft capsule is of sealed construction encapsulating the active agent. The soft capsule may be made by various processes, such as the plate process, the rotary die process, the reciprocating die process, and the continuous process.

Materials useful for forming the capsule of the osmotic pump dosage form are commercially available materials including gelatin, gelatin having a viscosity of about 5 to about 30 millipoises and a bloom strength up to about 150 grams; gelatin having a bloom value of about 160 to about 250; a composition comprising gelatin, glycerine, water and titanium dioxide; a composition comprising gelatin, erythrosin, iron oxide and titanium dioxide; a composition comprising gelatin, glycerine, sorbitol, potassium sorbate and titanium dioxide; a composition comprising gelatin, acacia, glycerin, and water; and the like, and a combination comprising at least one of the foregoing materials.

The semipermeable wall forming composition can be applied to the exterior surface of the capsule in laminar arrangement by molding, forming, air spraying, dipping or brushing with a semipermeable wall forming composition. Other techniques that can be used for applying the semipermeable wall are the air suspension procedure and the pan coating procedures. The air suspension procedure includes suspending and tumbling the capsule arrangement in a current of air and a semipermeable wall forming composition until the wall surrounds and coats the capsule. The procedure can be repeated with a different semipermeable wall forming composition to form a semipermeable laminated wall.

Exemplary solvents suitable for manufacturing the semipermeable wall include inert inorganic and organic solvents that do not adversely harm the materials, the capsule wall, the active agent, the thermo-responsive composition, the expandable member, or the final dispenser. Solvents for manufacturing the semipermeable wall may be aqueous solvents, alcohols, ketones, esters, ethers, aliphatic hydrocarbons, halogenated solvents, cycloaliphatics, aromatics, heterocyclic solvents, and a combination comprising at least one of the foregoing solvents. Particular solvents include acetone, diacetone alcohol, methanol, ethanol, isopropyl alcohol, butyl alcohol, methyl acetate, ethyl acetate, isopropyl acetate, n-butyl acetate, methyl isobutyl ketone, methyl propyl ketone, n-hexane, n-heptane, ethylene glycol monoethyl ether, ethylene glycol monoethyl acetate, methylene dichloride, ethylene dichloride, propylene dichloride, carbon tetrachloride, nitroethane, nitropropane, tetrachloroethane, ethyl ether, isopropyl ether, cyclohexane, cyclooctane, benzene, toluene, naphtha, 1,4-dioxane, tetrahydrofuran, water, and mixtures thereof such as acetone and water, acetone and methanol, acetone and ethyl alcohol, methylene dichloride and methanol, and ethylene dichloride, methanol, and a combination comprising at least one of the foregoing solvents. The semipermeable wall may be applied at a temperature a few degrees less than the melting point of the thermo-responsive composition. Alternatively, the thermo-responsive composition can be loaded into the dispenser after applying the semipermeable wall.

The exit means or hole in the osmotic pump dosage form, for releasing the active agent, can be formed by mechanical or laser drilling, or by eroding an erodible element in the wall, such as a gelatin plug. The orifice can be a polymer inserted into the semipermeable wall, which polymer is a porous polymer and has a pore, or which polymer is a microporous polymer and has a micro-pore.

Other extended-release formulations can include those that are easily administered for those patients that have difficulty with oral solid dosage formulations, such as tablets and capsules. Such formulations would be useful for the very young and elderly patients who require dosage forms that are easy to swallow. Easily administered formulations, such as chewable tablets, gummy forms, candy forms, sprinkle forms, liquid formulations (e.g. suspensions or emulsions), taste-masked formulations, and fast dissolve tablets, are thus desirable.

For easy administration, the extended-release form can be a chewable tablet containing quinine or a salt thereof. A chewable tablet comprises a chewable base and optionally a sweetener. The chewable base comprises an excipient such as, for example, mannitol, sorbitol, lactose, or a combination comprising at least one of the foregoing excipients. The optional sweetener used in the chewable dosage form may be, for example, digestible sugars, sucrose, liquid glucose, sorbitol, dextrose, isomalt, liquid maltitol, aspartame, lactose, and a combination comprising at least one of the foregoing sweeteners. In certain cases, the chewable base and the sweetener may be the same component. The chewable base and optional sweetener may comprise about 50 wt % to about 90 wt % of the total weight of the dosage form.

The chewable dosage form may additionally contain preservatives, agents that prevent adhesion to oral cavity and crystallization of sugars, flavoring agents, souring agents, coloring agents, and a combination comprising at least one of the foregoing agents. Glycerin, lecithin, hydrogenated palm oil or glyceryl monostearate may be used as a protecting agent of crystallization of the sugars in an amount of about 0.04 wt % to about 10 wt % of the total weight of the ingredients, to prevent adhesion to oral cavity and improve the soft property of the products. Additionally, isomalt or liquid maltitol may be used to enhance the chewing properties of the chewable dosage form.

Since quinine is bitter tasting, it can be taste-masked for better patient compliance. Quinine may be present in microparticles, wherein each microparticle incorporates quinine or a salt thereof in conjunction with a protective material. The microparticle may be provided as a microcapsule or as a matrix-type microparticle. Microcapsules may incorporate a discrete mass of quinine or a salt thereof surrounded by a discrete, separately observable coating of the protective material. Conversely, in a matrix-type particle, the quinine or a salt thereof is dissolved, suspended or otherwise dispersed throughout the protective material. Certain microparticles may include attributes of both microcapsules and matrix-type particle. For example, a microparticle may incorporate a core incorporating a dispersion of quinine or a salt thereof in a first protective material and a coating of a second protective material, which may be the same as or different from the first protective material surrounding the core. Alternatively, a microparticle may incorporate a core consisting essentially of quinine or a salt thereof and a coating incorporating the protective material, the coating itself having some of the quinine or a salt thereof dispersed within it. Specifically protective material can be a release-retarding material and/or taste-masking material.

The microparticles can have a mean outside diameter of up to about 600 micrometers, specifically about 75 to about and 500 micrometers, and more specifically about 150 to about 500 micrometers. Microparticles above about 200 micrometers may be used. Thus, the microparticles may be between about 200 mesh and about 30 mesh U.S. standard size, and more specifically between about 100 mesh and about 35 mesh.

Sprinkle dosage forms include particulate or pelletized forms of quinine or a salt thereof, optionally having functional or non-functional coatings, with which a patient or a caregiver can sprinkle the particulate/pelletized dose into drink or onto soft food. A sprinkle dosage form may comprise particles of about 10 to about 100 micrometers in their major dimension. Sprinkle dosage forms may be in the form of optionally coated granules or as microcapsules. Specifically the sprinkle dosage forms are extended-release formulations. See U.S. Pat. No. 5,084,278, which is hereby incorporated by reference for its teachings regarding microcapsule formulations, which may be administered as sprinkle dosage forms.

Another oral dosage form is a non-chewable, fast dissolving dosage form of quinine. These dosage forms can be made by methods known to those of ordinary skill in the art of pharmaceutical formulations. For example, Cima Labs has produced oral dosage forms including microparticles and effervescents, which rapidly disintegrate in the mouth and provide adequate taste-masking. Cima Labs has also produced a rapidly dissolving dosage form containing the active agent and a matrix that includes a nondirect compression filler and a lubricant. U.S. Pat. No. 5,178,878 and U.S. Pat. No. 6,221,392 provide teachings regarding fast-dissolve dosage forms.

An exemplary fast dissolve dosage form includes a mixture incorporating a water and/or saliva activated effervescent disintegration agent and microparticles. The microparticles can include those previously described for the chewable forms. The mixture including the microparticles and effervescent disintegration agent desirably may be present as a tablet of a size and shape adapted for direct oral administration to a patient. The tablet is substantially completely disintegrable upon exposure to water and/or saliva. The effervescent disintegration agent is present in an amount effective to aid in disintegration of the tablet, and to provide a distinct sensation of effervescence when the tablet is placed in the mouth of a patient.

The effervescent sensation is not only pleasant to the patient but also tends to stimulate saliva production, thereby providing additional water to aid in further effervescent action. Thus, once the tablet is placed in the patient's mouth, it will disintegrate rapidly and substantially completely without any voluntary action by the patient. Even if the patient does not chew the tablet, disintegration will proceed rapidly. Upon disintegration of the tablet, the microparticles are released and can be swallowed as a slurry or suspension of the microparticles. The microparticles thus may be transferred to the patient's stomach for dissolution in the digestive tract and systemic distribution of the pharmaceutical ingredient.

The term effervescent disintegration agent(s) includes compounds which evolve gas. The preferred effervescent agents evolve gas by means of chemical reactions which take place upon exposure of the effervescent disintegration agent to water and/or to saliva in the mouth. The bubble or gas generating reaction is most often the result of the reaction of a soluble acid source and an alkali metal carbonate or carbonate source. The reaction of these two general classes of compounds produces carbon dioxide gas upon contact with water included in saliva.

Such water activated materials may be kept in a generally anhydrous state with little or no absorbed moisture or in a stable hydrated form since exposure to water will prematurely disintegrate the tablet. The acid sources or acid may be those which are safe for human consumption and may generally include food acids, acid anhydrides and acid salts. Food acids include citric acid, tartaric acid, malic acid, fumaric acid, adipic acid, and succinic acids etc. Because these acids are directly ingested, their overall solubility in water is less important than it would be if the effervescent tablet formulations were intended to be dissolved in a glass of water. Acid anhydrides and acid of the above described acids may also be used. Acid salts may include sodium, dihydrogen phosphate, disodium dihydrogen pyrophosphate, acid citrate salts and sodium acid sulfite.

Carbonate sources include dry solid carbonate and bicarbonate salts such as sodium bicarbonate, sodium carbonate, potassium bicarbonate and potassium carbonate, magnesium carbonate and sodium sesquicarbonate, sodium glycine carbonate, L-lysine carbonate, arginine carbonate, amorphous calcium carbonate, and a combination comprising at least one of the foregoing carbonates.

The effervescent disintegration agent is not always based upon a reaction which forms carbon dioxide. Reactants which evolve oxygen or other gasses which are safe are also considered within the scope. Where the effervescent agent includes two mutually reactive components, such as an acid source and a carbonate source, it is preferred that both components react substantially completely. Therefore, an equivalent ratio of components which provides for equal equivalents is preferred. For example, if the acid used is diprotic, then either twice the amount of a mono-reactive carbonate base, or an equal amount of a di-reactive base should be used for complete neutralization to be realized. However, the amount of either acid or carbonate source may exceed the amount of the other component. This may be useful to enhance taste and/or performance of a tablet containing an overage of either component. In this case, it is acceptable that the additional amount of either component may remain unreacted.

In general, the amount of effervescent disintegration agent useful for the formation of tablets is about 5 wt % to about 50 wt % of the final composition, specifically about 15 wt % and about 30 wt % thereof, and most specifically about 20 wt % to about 25 wt % of the total composition.

Other fast dissolving quinine dosage forms can be prepared without an effervescent agent using spray dried carbohydrate or sugar alcohol excipients (e.g. sorbitol, mannitol, xylitol, a combination comprising at least one of the foregoing, and the like), optionally combined with a disintegrant (e.g. the disintegrant is selected from crospovidone, croscarmellose, sodium starch glycolate, a combination comprising at least one of the foregoing, and the like), and/or a glidant (e.g. colloidal silica, silica gel, precipitated silica, a combination comprising at least one of the foregoing, and the like). Suitable fast-dissolve can be found in U.S. Patent Application Publication US20030118642 A1 to Norman et al. incorporated herein in its entirety.

The tablets of a fast dissolving dosage form should rapidly disintegrate when orally administered. By “rapid”, it is understood that the tablets should disintegrate in the mouth of a patient in less than about 10 minutes, and desirably between about 30 seconds and about 7 minutes, specifically the tablet should dissolve in the mouth between about 30 seconds and about 5 minutes. Disintegration time in the mouth can be measured by observing the disintegration time of the tablet in water at about 37° C. The tablet is immersed in the water without forcible agitation. The disintegration time is the time from immersion for substantially complete dispersion of the tablet as determined by visual observation. As used herein, the term “complete disintegration” of the tablet does not require dissolution or disintegration of the microcapsules or other discrete inclusions.

Fast-dissolve tablets can be manufactured by well-known tableting procedures. In common tableting processes, the material which is to be tableted is deposited into a cavity, and one or more punch members are then advanced into the cavity and brought into intimate contact with the material to be pressed, whereupon compressive force is applied. The material is thus forced into conformity with the shape of the punches and the cavity. Hundreds, and even thousands, of tablets per minute can be produced in this fashion.

Taste-masked solid dosage forms of quinine are useful since quinine exhibits a particularly bitter taste. A solid taste-masked dosage form comprises a core element comprising quinine or a salt thereof and a coating surrounding the core element. The core element comprising quinine or a salt thereof may be in the form of a capsule or be encapsulated by micro-encapsulation techniques, where a polymeric coating is applied to the formulation. The core element can also include excipients, fillers, flavoring agents, stabilizing agents and/or colorants.

The taste-masked dosage form may include about 77 wt % to about 100 wt %, specifically about 80 wt % to about 90 wt %, based on the total weight of the composition of a core element comprising quinine or a salt thereof; and about 20 wt % to about 70 wt %, of a substantially continuous coating on the core element formed from a coating material including a polymer. The core element includes about 52 wt % to about 85 wt % of quinine or a salt thereof; and approximately 5 wt % to about 25 wt % of a supplementary component selected from waxes, water insoluble polymers, enteric polymers, and partially water soluble polymers, other suitable pharmaceutical excipients, and a combination comprising at least one of the foregoing components.

The coating material of the taste-masked formulation may take a form which provides a substantially continuous coating and still provides taste-masking. In some cases, the coating also provides controlled-release of the active agent. The polymer used in taste-masked dosage form coating may be a water insoluble polymer such as, for example, ethyl cellulose. The coating material of the taste-masked dosage form may further include a plasticizer.

A method of preparing taste-masked pharmaceutical formulations such as powdered formulations includes mixing a core element and a coating material in a diluent and spray drying the mixture to form a taste-masked formulation. Spray drying of the active agent and polymer in the solvent involves spraying a stream of air into an atomized suspension so that solvent is caused to evaporate leaving the active agent coated with the polymer coating material.

Liquid dosage forms of quinine or a salt thereof may be formulated to provide adequate taste-masking as well as extended-release properties. A taste-masked liquid dosage form may comprise a suspension of taste-masked particles (e.g., microparticles). The use of polymeric coatings on the active agent microparticles, which inhibit or retard the rate of dissolution and solubilization of the active agent is one means of overcoming the taste problems with delivery of active agents in suspension. The polymeric coating allows time for all of the particles to be swallowed before the taste threshold concentration is reached in the mouth.

A taste-masked liquid dosage form thus comprises the active agent, a polymer encapsulating the active agent, and a suspending medium for suspending the encapsulated active agent. The active agent can be taste-masked by the polymer or polymer and suspending medium.

The quinine may be in the form of its neutral or salt form and may be in the form of particles, crystals, microcapsules, granules, microgranules, powders, pellets, amorphous solids or precipitates. The particles may further include other functional components. The quinine particles may have a defined particle size distribution, specifically in the region of less than or equal to about 1000 micrometers, specifically less than or equal to about 750 micrometers, more specifically less than or equal to about 500 micrometers, yet more specifically less than or equal to about 250 micrometers, and still yet more specifically less than or equal to about 150 micrometers, where there is acceptable mouth feel and little chance of chewing on the residual particles and releasing the active agent to taste.

The taste-masked liquid dosage form may include, along with quinine or a salt thereof, other functional components present for the purpose of modifying the physical, chemical, or taste properties of the quinine. For example the quinine may be in the form of ion-exchange or cyclodextrin complexes or the quinine may be included as a mixture or dispersion with various additives such as waxes, lipids, dissolution inhibitors, taste-masking or -suppressing agents, carriers or excipients, fillers, and a combination comprising at least one of the foregoing components. When used in such taste-masked formulations, the size of the quinine salt particle can be of any size, from the molecular level, up to about smicrometer size.

The pharmaceutically active agent or the active agent particle may be suspended, dispersed or emulsified in the suspending medium after encapsulation with the polymer. The suspending medium may be a water-based medium, but may be a non-aqueous carrier as well. The taste-masked liquid dosage form may further include other optional dissolved or suspended agents to provide stability to the suspension. These include suspending agents or stabilizers such as, for example, methyl cellulose, sodium alginate, xanthan gum, (poly)vinyl alcohol, microcrystalline cellulose, colloidal silicas, bentonite clay, and a combination comprising at least one of the foregoing agents. Other agents used include preservatives such as methyl, ethyl, propyl and butyl parabens, sweeteners such as sucrose, saccharin sodium, aspartame, mannitol, flavorings such as grape, cherry, peppermint, menthol and vanilla flavors, and antioxidants or other stabilizers, and a combination comprising at least one of the foregoing agents.

Encapsulation of the microparticle or active agent particle by the polymer may be performed by a method such as suspending, dissolving, or dispersing in a solution or dispersion of polymer coating material and spray drying, fluid-bed coating, simple or complex coacervation, coevaporation, co-grinding, melt dispersion and emulsion-solvent evaporation techniques, and the like.

The polymer coated quinine, or salt thereof, powder can also as an alternative be applied for the preparation of reconstitutable powders, ie; dry powder active agent products that are reconstituted as suspensions or emulsions in a liquid vehicle such as water before usage. The reconstitutable powders have a long shelf life and the suspensions, once reconstituted, have adequate taste-masking.

Suitable liquid taste-masked dosage forms include those disclosed in U.S. Pat. No. 6,197,348.

The quinine or pharmaceutically acceptable salt thereof can also be formulated into parenteral depot formulations. Parenteral depot formulations are injected or implanted into the muscle or subcutaneous tissue and release quinine in a controlled manner. An advantage of depot forms is the sustained-release of quinine for several days or weeks.

Such forms can be in the form of microparticles or implants (e.g., rod-shaped). Implants are rod-shaped devices injected through a large bore needle into the subcutaneous tissue. Microparticies are generally spherical and can be injected intramuscularly or subcutaneously as their size typically range from about 1 to about 1000 micrometers, specifically about 10 to about 100 micrometers. Microparticles can include i) microcapsules, that is microparticles containing quinine in a core surrounded by a polymeric membrane; and ii) microspheres, that is microparticles containing the drug in a polymeric matrix, forming a solid dispersion or solid solution.

The depot formulations can be prepared from biodegradable polymer excipients or non-biodegradable polymer excipients. The polymer excipient controls the rate of drug release and, if biodegradable, resorbs during and/or after drug release.

Exemplary biodegradable polymers are lactide/glycolide polymers, while an exemplary non-biodegradable polymer is ethylene vinylacetate copolymer. Overall drug release may be controlled by varying the polymer composition. For example, an increase in the level of lactic acid in a lactide/glycolide polymer can retard drug release and an increase in the polymer molecular weight also can retard drug release and prolong drug effects in vivo.

Exemplary extended-release forms are described in U.S. Pat. No. 5,102,666 incorporated herein by reference. As described therein a polymeric composition comprises a reaction complex formed by the interaction of (1) a calcium polycarbophil component which is a water-swellable, but water insoluble, fibrous cross-linked carboxy-functional polymer, the polymer containing (a) a plurality of repeating units of which at least about 80% contain a carboxyl functionality, and (b) about 0.05 to about 1.5% cross-linking agent substantially free from polyalkenyl polyether, the percentages being based upon the weights of unpolymerized repeating unit and cross-linking agent, respectively, with (2) water, in the presence of an active agent.

The amount of calcium polycarbophil present can be about 0.1 to about 99% by weight, for example about 10%. The amount of quinine or a salt thereof present can be about 0.0001 to about 65% by weight, for example about 5 to about 20% of the reaction complex. The amount of water present can be about 5 to about 200% by weight, for example about 5 to about 10%. The interaction is carried out at a pH of between about 3 and about 10, for example about 6 to 7. The calcium polycarbophil is originally present in the form of a calcium salt containing about 5 to about 25% calcium.

Several types of materials are suitable for forming the polycarbophil type composition component. The polymer contains a plurality of a repeating unit of which at least about 80 percent contain a carboxyl functionality and about 0.05 to about 1.5 percent cross-linking agent substantially free from polyalkenyl polyether, with the percentages being based upon the weights of the unpolymerized repeating unit and cross-linking agent, respectively. Specifically, at least about 90 percent of the repeating units contain a carboxyl functionality, and more specifically, at least 95 percent of those repeating units contain a carboxyl functionality. Still yet more specifically, this material is a reaction product of the polymerization of only a carboxyl-functional monomer and a cross-linking agent. More specifically, this component contains about 0.1 to about 1 percent by weight of polymerized cross-linking agent. The material also contains from 5% to 25%, specifically 18% to 22% calcium as a calcium salt of the polymer acid. Certain species of this type of polymer is commercially available under the generic name “calcium polycarbophil”.

A calcium polycarbophil type composition polymer useful herein may thus be defined as a reaction product of the copolymerization of at least 80 weight percent monoethylenically unsaturated carboxy-functional monomer and about 0.05 to about 1.5 weight percent of a cross-linking agent free of polyalkenyl polyether and 18-22% of calcium.

In addition to the above two ingredients, the polycarbophil type polymer may also include polymerized monoethylenically unsaturated repeating units such as C₁-C₆ alkyl esters of one or more of the above-described acids such as hexyl acrylate, butyl methacrylate and methyl crotonate; hydroxyalkylene-functional esters of the above-described acids that contain a per molecule average of 1 to about 4 oxyalkylene groups containing 2-3 carbon atoms such as hydroxyethyl methacrylate, hydroxypropyl acrylate and tetraethylene glycol monoacrylate; methacrylamide, acrylamide and their C₁-C₄ mono- and dialkyl derivatives such as N-methyl acrylamide, N-butyl methacrylamide and N,N-dimethyl acrylamide; styrene; and the like as are known in the art as being copolymerizable with the above described carboxyl functionality-containing monomers and cross-linking agents. The polymers most specifically are prepared from only the monoethylenically unsaturated carboxy-functional monomer and the cross-linking agent.

The interaction of the calcium polycarbophil with the water results in the formation of a complex hydrogel matrix structure which then acts to control the diffusion or other transport of the quinine or salt thereof within and from the matrix itself. The desired level of controlled or sustained-release will vary, depending upon the ratio of the components employed, the physical state of the quinine or salt thereof, the method of incorporation, the order of mixing of the components, and the like. Additional additives may also be present which may modify the characteristics of the matrix and its release properties.

In one embodiment, an extended-release formulation comprises a polymeric composition comprising a reaction complex formed by the interaction of water and a calcium polycarbophil component; wherein the calcium polycarbophil component is a water-swellable, but water insoluble, fibrous cross-linked carboxy-functional polymer comprising (a) a plurality of repeating units of which at least about 80% contain a carboxyl functionality, and (b) about 0.05 to about 1.5% cross-linking agent substantially free from polyalkenyl polyether, the percentages being based upon the weights of unpolymerized repeating unit and cross-linking agent, respectively; and wherein the reaction complex is formed in the presence of quinine or a pharmaceutically acceptable salt thereof.

In another embodiment, a process for preparing an extended-release formulation comprises combining, in the presence of quinine or a pharmaceutically acceptable salt thereof, water and a calcium polycarbophil component; wherein the calcium polycarbophil component is a water-swellable, but water insoluble, fibrous cross-linked carboxy-functional polymer, the fibrous cross-linked carboxy-functional polymer comprises (a) a plurality of repeating units of which at least about 80% contain a carboxyl functionality, and (b) about 0.05 to about 1.5% cross-linking agent substantially free from polyalkenyl polyether, the percentages being based upon the weights of unpolymerized repeating unit and cross-linking agent, respectively.

Additional exemplary extended-release forms are described in U.S. Pat. No. 5,422,123 incorporated herein by reference. Described therein are tablets consisting of a core of defined geometrical form containing an active substance, polymer substances which swell on contact with aqueous liquids, substances with gelling properties, and possibly other substances with an adjuvant function; and a support applied to the core to partly cover its surface, and are characterized in that the support consists of polymer substances which are slowly soluble and/or slowly gellable in aqueous liquids, plasticizing substances, and possibly other substances with an adjuvant function, which plasticizing action can also be performed by the polymer substances.

The core can be prepared by compressing the core mixture containing quinine or a salt thereof under a pressure of about 1000 to about 4000 kg/cm² and therefore assumes a defined geometrical form. Exemplary forms include a cylindrical tablet with flat, convex, or concave bases.

Polymer materials suitable to prepare the core are those which swell on contact with aqueous liquids, essentially insoluble polymers are used such as crosslinked sodium carboxymethylcellulose, crosslinked hydroxypropylcellulose, high molecular weight hydroxypropylmethylcellulose, carboxymethyl starch, potassium methacrylate/divinylbenzene copolymer, polymethylmethacrylate, crosslinked polyvinylpyrrolidone, high molecular weight polyvinylalcohols etc. Gellable polymer materials include methylcellulose, carboxymethylcellulose, low molecular weight hydroxypropylmethylcellulose, low molecular weight polyvinylalcohols, polyethylene glycols, non-crosslinked polyvinylpyrrolidone. Polymers which possess both swelling and gelling properties such as medium viscosity hydroxypropylmethylcellulose and medium viscosity polyvinylalcohols can also be used. Adjuvant substances include mannitol, ethylcellulose, magnesium stearate, colloidal silica and others.

The ratio of polymer substances with swelling properties to gellable polymer substances is between about 1:9 to about 9:1. The active agent content in the core can be about 1 to about 95% by weight based on the total weight of the core.

The support generally has a thickness of about 10 micrometers to about 4 millimeters depending on the hydrophilic characteristics of the components, its task being to limit and define the direction of release of the active substance contained in the core. As the support is generally less hydrophilic than the core and does not contain active agent, the transfer of active agent can occur to a significant and immediate extent only from that portion of the core which is not covered by the support.

Suitable materials that can be used to prepare the support include support polymer substances slowly soluble and/or slowly gellable in aqueous liquids, these substances being used either alone or in mixture with each other, are chosen from the group consisting of hydroxypropylmethylcellulose having a molecular weight of about 4,000 to about 2,000,000, high molecular weight carboxyvinylpolymers, polyvinylalcohols, scleroglucans, acrylates, methacrylates, hydroxypropylcellulose, sodium carboxymethylcellulose, and hydrophilic cellulose derivatives.

The support polymer substances are present in about 2 to about 95 weight % and specifically about 30 to about 90 weight % of the support composition. The support composition also includes substances able to provide elasticity, such as polyethylene glycols, castor oil, hydrogenated castor oil, ethyl phthalate, butyl phthalate, and natural, synthetic and semisynthetic glycerides, and the like. The support elasticity substances ensure correct release kinetics, determined by the fact that the support is sufficiently elastic to follow any change consequent on the hydration of the core without causing cracking or gaps which would result in total, and premature, release of the active agent.

These support elasticity substances can be present in zero to about 50 weight % and specifically about 2 to about 15 weight % of the total weight of the support.

Finally, the support composition can include binders such as polyvinylpyrrolidone, methylcellulose, ethylcellulose, gum arabic, alginic acid and its derivatives, hydrophilic agents such as mannitol, lactose, starch, colloidal silica, and hydrophobic agents such as hydrogenated castor oil, magnesium stearate, fatty substances, waxes, and natural and synthetic glycerides. Choice of hydrophilic and hydrophobic agents controls the hydrophilic properties of the support and the desired release rate. The binders, hydrophilic agents and hydrophobic agents can be present in an amount of about zero to about 50 weight and specifically about 0.5 to about 35 weight % of the total weight of the support.

The components of the support are prepared by mixing, possibly wetting with a binding solution in accordance with the known art, then bringing the mixture to the dry granular state. The mixture can be screened and mixed with other components until an easily flowable homogeneous mixture is obtained. The prepared support mixture is then applied to the core as a surface layer by using presses. The support can be applied to one or two bases of the core, or can be applied to the entire core surface with the exception of one base, or to the entire lateral surface with the exclusion of the two bases. The support is typically applied using a pressure of about 1000 to about 4000 kg/cm².

In one embodiment, an extended-release formulation comprises (a) a deposit-core having a defined geometric form and comprising a therapeutically effective amount of quinine or a pharmaceutically acceptable salt thereof, and a core polymeric material selected from the group consisting of (1) a swellable polymeric material which swells on contact with water or aqueous liquids and a gellable polymeric material, wherein the ratio of the swellable polymeric material to gellable polymeric material is about 1:9 to about 9:1, and (2) a single polymeric material having both swelling and gelling properties; and (b) a support-platform applied to the deposit-core, and wherein the support-platform is an elastic support applied to the deposit-core so that it partially covers a surface of the deposit-core and follows changes due to hydration of the deposit-core and is slowly soluble and/or slowly gellable in aqueous fluids.

The support-platform of this embodiment can comprise a polymer substance which is slowly soluble or slowly gellable in aqueous liquids and a plasticizing substance. The plasticizing substance contained in the support-platform can be selected from the group consisting of polyoxyethylene glycols, castor oil, hydrogenated castor oil, ethyl phthalate, butyl phthalate, natural glycerides, synthetic glycerides, and semisynthetic glycerides. The plasticizing substance can be present at about 2 to about 15% by weight of the total weight of the support-platform.

Furthermore, in this embodiment, the support-platform can further comprise a binder selected from the group consisting of polyvinylpyrrolidone, methylcellulose, ethylcellulose, gum arabic, and alginic acid. The support-platform can comprise a hydrophilic agent selected from the group consisting of mannitol, lactose, starch, and colloidal silica. The support-platform can comprise a hydrophobic agent selected from the group consisting of hydrogenated castor oil, magnesium stearate, a fatty substance, wax, natural glycerides, and synthetic glycerides.

Furthermore in this embodiment, the core polymeric material can be selected from the group consisting of crosslinked sodium carboxymethylcellulose, crosslinked hydroxypropylcellulose, high molecular weight hydroxypropylmethylcellulose, carboxymethyl starch, potassium methacrylate/divinylbenzene copolymer, polymethylmethacrylate, crosslinked polyvinylpyrrolidone, high molecular weight polyvinylalcohols, methylcellulose, carboxymethylcellulose, low molecular weight hydroxypropylmethylcellulose, low molecular weight polyvinylalcohols, polyethylene glycols, non-crosslinked polyvinylpyrrolidone, medium viscosity hydroxypropylmethylcellulose, medium viscosity polyvinylalcohols, and a combination comprising at least one of the foregoing.

In another embodiment, a process for preparing an extended-release formulation comprises granulating deposit-core ingredients to form a core granular mixture, wherein the deposit-core ingredients comprise a therapeutically effective amount of quinine or a pharmaceutically acceptable salt thereof, and a core polymeric material selected from the group consisting of (1) a swellable polymeric material which swells on contact with water or aqueous liquids and a gellable polymeric material, wherein the ratio of the swellable polymeric material to gellable polymeric material is about 1:9 to about 9:1, and (2) a single polymeric material having both swelling and gelling properties; compressing the core granular mixture to form a deposit-core of a defined geometrical form; screening and mixing support-platform components to obtain a support granular mixture, wherein the support-platform components comprise a polymer substance which is slowly soluble or slowly gellable in aqueous liquids, and a plasticizing substance; and applying the support granular mixture onto a portion of a surface of the deposit-core by compressing to form the support-platform partially covering the deposit-core of defined geometrical form.

As used herein, “pharmaceutically acceptable excipient” means any other component added to the pharmaceutical formulation other than the active agent. Excipients may be added to facilitate manufacture, enhance stability, control release, enhance product characteristics, enhance bioavailability, enhance patient acceptability, etc. Pharmaceutical excipients include carriers, fillers, binders, disintegrants, lubricants, glidants, compression aids, colors, sweeteners, preservatives, suspending agents, dispersing agents, film formers, flavors, printing inks, etc.

Binders hold the ingredients in the dosage form together. Exemplary binders include, for example, polyvinyl pyrrolidone, hydroxypropyl cellulose, hydroxypropyl methylcellulose, methylcellulose and hydroxyethyl cellulose, sugars, and a combination comprising at least one of the foregoing binders.

Disintegrants expand when wet causing a tablet to break apart. Exemplary disintegrants include water swellable substances, for example, low-substituted hydroxypropyl cellulose, e.g. L-HPC; cross-linked polyvinyl pyrrolidone (PVP-XL), e.g. Kollidon® CL and Polyplasdone® XL; cross-linked sodium carboxymethylcellulose (sodium croscarmellose), e.g. Ac-di-sol®, Primellose®; sodium starch glycolate, e.g. Primojel®; sodium carboxymethylcellulose; sodium carboxymethyl starch, e.g. Explotab®; ion-exchange resins, e.g. Dowex® or Amberlite®; microcrystalline cellulose, e.g. Avicel®; starches and pregelatinized starch, e.g. Starch 1500®; formalin-casein, and a combination comprising at least one of the foregoing water swellable substances.

Lubricants, for example, aid in the processing of powder materials. Exemplary lubricants include calcium stearate, glycerol behenate, magnesium stearate, mineral oil, polyethylene glycol, sodium stearyl fumarate, stearic acid, talc, vegetable oil, zinc stearate, and a combination comprising at least one of the foregoing lubricants. Glidants include, for example, silicon dioxide.

Certain dosage forms described herein contain a filler, such as a water insoluble filler, water soluble filler, and a combination comprising at least one of the foregoing. The filler may be a water insoluble filler, such as silicon dioxide, titanium dioxide, talc, alumina, starch, kaolin, polacrilin potassium, powdered cellulose, microcrystalline cellulose, and a combination comprising at least one of the foregoing fillers. Exemplary water-soluble fillers include water soluble sugars and sugar alcohols, specifically lactose, glucose, fructose, sucrose, mannose, dextrose, galactose, the corresponding sugar alcohols and other sugar alcohols, such as mannitol, sorbitol, xylitol, and a combination comprising at least one of the foregoing fillers.

The dosage form can be prepared by various conventional mixing, comminution and fabrication techniques readily apparent to those skilled in the art of drug formulations. Examples of such techniques include direct compression, using appropriate punches and dies, the punches and dies are fitted to a suitable rotary tableting press; injection or compression molding using suitable molds fitted to a compression unit, granulation followed by compression; and extrusion in the form of a paste, into a mold or to an extrudate to be cut into lengths.

Oral dosage forms may be prepared to include an effective amount of melt-extruded subunits in the form of multiparticles within a capsule. For example, a plurality of the melt-extruded muliparticulates can be placed in a gelatin capsule in an amount sufficient to provide an effective release dose when ingested and contacted by gastric fluid.

The subunits, e.g., in the form of multiparticulates, can be compressed into an oral tablet using conventional tableting equipment using standard techniques. Techniques and compositions for making tablets (compressed and molded), capsules (hard and soft gelatin) are also described in Remington's Pharmaceutical Sciences, (Aurther Osol., editor), 1553-1593 (1980).

The composition may be in the form of micro-tablets enclosed inside a capsule, e.g. a gelatin capsule. For this, a gelatin capsule employed in the pharmaceutical formulation field can be used, such as the hard gelatin capsule known as CAPSUGEL, available from Pfizer.

Certain dosage forms described herein may be coated. The coating can be a suitable coating, such as, a functional or a non-functional coating, or multiple functional and/or non-functional coatings. By “functional coating” is meant to include a coating that modifies the release properties of the total formulation, for example, an extended-release coating. By “non-functional coating” is meant to include a coating that is not a functional coating, for example, a cosmetic coating. A non-functional coating can have some impact on the release of the active agent due to the initial dissolution, hydration, perforation of the coating, etc., but would not be considered to be a significant deviation from the non-coated composition.

The dosage forms described herein may be coated with a functional or non-functional coating. The coating may comprise about 0 wt % to about 40 wt % of the composition. The coating material may include a polymer, specifically a film-forming polymer, for example, methyl cellulose, ethyl cellulose, hydroxypropyl cellulose, hydroxypropyl methyl cellulose, hydroxybutyl methyl cellulose, cellulose acetate, cellulose propionate, cellulose acetate propionate, cellulose acetate butyrate, cellulose acetate phthalate, carboxymethyl cellulose, cellulose triacetate, cellulose sulphate sodium salt, poly(methyl methacrylate), poly(ethyl methacrylate), poly(butyl methacrylate), poly(isobutyl methacrylate), poly(hexyl methacrylate), poly(phenyl methacrylate), poly(methyl acrylate), poly(isopropyl acrylate), poly(isobutyl acrylate), poly(octadecyl acrylate), poly(ethylene), poly(ethylene) low density, poly (ethylene) high density, (poly propylene), poly(ethylene glycol, poly(ethylene oxide), poly(ethylene terephthalate), poly(vinyl alcohol), poly(vinyl isobutyl ether), poly(viny acetate), poly(vinyl chloride), polyvinyl pyrrolidone, and a combination comprising at least one of the foregoing polymers.

To provide a taste-masking effect, the polymer can be a water-insoluble polymer. Water insoluble polymers include ethyl cellulose or dispersions of ethyl cellulose, acrylic and/or methacrylic ester polymers, cellulose acetates, butyrates or propionates or copolymers of acrylates or methacrylates having a low quaternary ammonium content, and the like, and a combination comprising at least one of the foregoing polymers.

The inclusion of an effective amount of a plasticizer in the coating composition can improve the physical properties of the film. For example, because ethyl cellulose has a relatively high glass transition temperature and does not form flexible films under normal coating conditions, it may be advantageous to add plasticizer to the ethyl cellulose before using the same as a coating material. Generally, the amount of plasticizer included in a coating solution is based on the concentration of the polymer, e.g., most often from about 1 wt % to about 50 wt % of the polymer. Concentrations of the plasticizer, however, can be determined by routine experimentation.

Examples of plasticizers for ethyl cellulose and other celluloses include plasticizers such as dibutyl sebacate, diethyl phthalate, triethyl citrate, tributyl citrate, triacetin, and a combination comprising at least one of the foregoing plasticizers, although it is possible that other water-insoluble plasticizers (such as acetylated monoglycerides, phthalate esters, castor oil, etc.) can be used.

Examples of plasticizers for acrylic polymers include citric acid esters such as triethyl citrate NF, tributyl citrate, dibutyl phthalate, 1,2-propylene glycol, polyethylene glycols, propylene glycol, diethyl phthalate, castor oil, triacetin, and a combination comprising at least one of the foregoing plasticizers, although it is possible that other plasticizers (such as acetylated monoglycerides, phthalate esters, castor oil, etc.) can be used.

An example of a functional coating comprises a coating agent comprising a poorly-water-permeable component (a) such as, an alkyl cellulose, for example an ethylcellulose, such as AQUACOAT (a 30% dispersion available from FMC, Philadelphia, Pa.) or SURELEASE (a 25% dispersion available from Colorcon, West Point, Pa.) and a water-soluble component (b), e.g., an agent that can form channels through the poorly-water-permeable component upon the hydration or dissolution of the soluble component. Specifically, the water-soluble component is a low molecular weight, polymeric material, e.g., a hydroxyalkylcellulose, hydroxyalkyl(alkylcellulose), and carboxymethylcellulose, or salts thereof. Particular examples of these water soluble polymeric materials include hydroxyethylcellulose, hydroxypropylcellulose, hydroxyethylmethylcellulose, hydroxypropylmethylcellulose, carboxymethylcellulose, sodium carboxymethylcellulose, and a combination comprising at least one of the foregoing materials. The water-soluble component can comprise hydroxypropylmethylcellulose, such as METHOCEL (Dow). The water-soluble component can be of relatively low molecular weight, specifically less than or equal to about 25,000 molecular weight, or specifically less than or equal to about 21,000 molecular weight.

In the functional coating, the total of the water soluble portion (b) and poorly-water permeable portion (a) are present in weight ratios (b):(a) of about 1:4 to about 2:1, specifically about 1:2 to about 1:1, and more specifically in a ratio of about 2:3. While the ratios disclosed herein are preferred for duplicating target release rates of presently marketed dosage forms, other ratios can be used to modify the speed with which the coating permits release of the active agent. The functional coating may comprise about 1 wt % to about 40 wt %, specifically about 3 wt % to about 30 wt %, more specifically about 5 wt % to about 25 wt %, and yet more specifically about 6 wt % to about 15 wt % of the total formulation.

Suitable methods can be used to apply the coating to the dosage forms. Processes such as simple or complex coacervation, interfacial polymerization, liquid drying, thermal and ionic gelation, spray drying, spray chilling, fluidized bed coating, pan coating, electrostatic deposition, may be used.

The coatings may be of any thickness, specifically about 0.005 micrometers to about 25 micrometers thick and more specifically about 0.05 micrometers to about 5 micrometers.

As disclosed herein, the exemplary dosage forms (e.g. containing extended-release quinine particles) exhibit a pharmacokinetic profile that has a fast onset and level peak and trough values. The dosage forms can be formulated to provide a dissolution profile that is substantially pH independent or, alternatively, pH dependent (e.g. enteric coated forms).

In one embodiment, the dosage form exhibits a dissolution profile such that at 60 minutes after combining the dosage form with 900 ml of purified water at 37° C.±0.5° C. according to USP 28<711> test method 2 (paddle), 75 rpm paddle speed, about 20 to about 40 weight percent of the total amount of quinine is released, and wherein after 10 hours greater than or equal to about 80% of the total amount of quinine is released.

In another embodiment, the dosage form exhibits a dissolution profile such that at 60 minutes after combining the dosage form with 900 ml of purified water at 37° C.±0.5° C. according to USP 28<711> test method 2 (paddle), 75 rpm paddle speed, about 10 to about 30 weight percent of the total amount of quinine is released, and wherein after 10 hours greater than or equal to about 70% of the total amount of quinine is released.

In yet another embodiment, the dosage form exhibits a dissolution profile such that at 2 hours after combining the dosage form with 0.1 N Hydrochloric Acid medium at 37° C.±0.5° C. according to USP 28<711> test method 1 or 2, about 0 to about 10 weight percent of the total amount of quinine is released and wherein after 2 hours when the medium is switched to a buffer phase of pH 4.5, 6.8, 7.0 or water, about 0 to about 100 weight percent of the total amount of quinine is released.

In another embodiment, the dosage form exhibits a dissolution profile such that at 2 hours after combining the dosage form with 0.1 N Hydrochloric Acid medium at 37° C.±0.5° C. according to USP 28<711> test method 1 or 2, about 0 to about 50 weight percent of the total amount of quinine is released and wherein after 2 hours when the medium is switched to a buffer phase of pH 4.5, 6.8, 7.0 or water, about 0 to about 100 weight percent of the total amount of quinine is released.

In another embodiment, the extended-release quinine formulation can reach Tₘₐₓ at about 1.5 to about 8 hours, specifically about 3 to about 7 hours, and more specifically about 5 to about 6 hours. After dosing an extended-release quinine formulation containing about 300-600 mg quinine the Cₘₐₓ is about 200 to about 7000 ng/mL, specifically about 500 to 5000 ng/mL, and more specifically about 1000 to about 3000 ng/mL; and the Cₘᵢₙ is about 100 to about 3500 ng/mL at 12 to 24 hours, when at steady state.

In yet another embodiment, the extended-release quinine formulation exhibits a pharmacokinetic profile wherein the duration of 50% or greater of Cₘₐₓ is about 10 to about 20 hours. Furthermore, the extended-release quinine formulation exhibits a pharmacokinetic profile wherein the duration of 80% or greater of Cₘₐₓ is about 2 to about 12 hours.

The formulations according deliver a therapeutically effective amount of quinine to a patient during the 16, specifically 18, and more specifically 24 hours following a single once daily administration.

In one embodiment, the extended-release quinine formulation exhibits greater bioavailability than a corresponding immediate-release formulation. Therefore, the extended-release formulation allows for the use of lower amounts of active agent while exhibiting the same bioequivalence as higher doses found in immediate-release forms.

In one embodiment, an extended-release quinine solid oral dosage form can comprise about 50 to about 1000 mg of quinine, more specifically about 100 to about 750 mg of quinine, and yet more specifically about 250 to about 500 mg of quinine base equivalent per dosage unit.

In one embodiment, an extended-release quinine solid oral dosage form can comprise about 350 to about 520 mg of quinine, more specifically about 450 to about 500 mg of quinine, and yet more specifically about 475 to about 490 mg of quinine base equivalent per dosage unit taken as two units three times a day, two or three units twice a day, or three or four units once a day.

In another embodiment, an extended-release quinine solid oral dosage form can comprise about 100 to about 400 mg of quinine, more specifically about 150 to about 350 mg of quinine, and yet more specifically about 200 to about 300 mg of quinine base equivalent per dosage unit taken as one, two, three, or four units once, twice, or three times a day.

In yet another embodiment, an extended-release quinine solid oral dosage form can comprise about 200 to about 600 mg of quinine sulfate, more specifically about 260 to about 520 mg of quinine sulfate, and yet more specifically about 300 to about 450 mg of quinine sulfate per dosage unit.

Also included herein are pharmaceutical kits useful, for example, for the treatment of parasitic diseases (e.g. uncomplicated Plasmodium falciparum malaria, severe or complicated Plasmodium falciparum malaria) caused by Plasmodium species (e.g. sp. Falciparum, Plasmodium falciparum), the treatment and prophylaxis of leg cramps, or the treatment of babesiosis caused by Babesia microti, which comprise one or more containers containing an extended-release form of quinine or a salt thereof. The kits may further comprise one or more conventional pharmaceutical kit components, such as, for example, one or more containers to aid in facilitating compliance with a particular dosage regimen; one or more carriers; printed instructions, either as inserts or as labels, indicating quantities of the components to be administered, and/or guidelines for administration. Exemplary kits can be in the form of bubble or blister pack cards, optionally arranged in a desired order for a particular dosing regimen. Suitable blister packs that can be arranged in a variety of configurations to accommodate a particular dosing regimen are well known in the art or easily ascertained by one of ordinary skill in the art.

In one embodiment, the controlled-release quinine formulation is packaged with information warning that quinine may cause QT/QTc prolongation as an adverse reaction in some patients.

Those forms existing as liquids, solutions, emulsions, or suspensions can be packaged for convenient dosing of pediatric or geriatric patients. For example, prefilled droppers (such as eye droppers or the like), prefilled syringes, and similar containers housing the liquid, solution, emulsion, or suspension form of the extended-release quinine formulation are contemplated.

In one embodiment, when the controlled-release quinine formulation comprises carboxy vinyl polymer, the formulation is free of polyethylene glycol, specifically a polyethylene glycol having a molecular weight of about 900 to about 25,000. In another embodiment, the controlled-release quinine formulation is free of a polymer or crosslinker comprising thiol groups. In yet another embodiment, the controlled-release quinine formulation is free of the combination of a low molecular weight polyethylene oxide (e.g. from about 100,000 to about 900,000), a high molecular weight polyethylene oxide (e.g. MW from about 1,000,000 to about 9,000,000) and a starch or starch derivative. In still yet another embodiment, when the controlled-release quinine formulation contains a biodegradable polymer, the formulation is free of chemotherapeutic agents. In still yet another embodiment, the controlled-release quinine formulation is free of Eudragit RS, a copolymer of acrylic acid and methacrylic acid esters containing about 4 to about 7% ammonio groups.

In yet another embodiment, the controlled-release quinine formulation contains no microcapsules encapsulated, coated, or surrounded by an anionic or cationic polymer. In one embodiment, the controlled-release quinine formulation contains only a controlled-release portion and no immediate-release portion. In one embodiment, the controlled-release quinine formulation comprises (meth)acrylic and (meth)acrylate copolymers and polymers that are free of tertiary amino groups. In one embodiment, the controlled-release quinine formulation is free of pectin. In another embodiment, the controlled-release quinine formulation is free of a copolymer of polyvinyl alcohol and (meth)acrylic acid.

In still yet another embodiment, the controlled-release quinine formulation is free of a hydroxypropyl methylcellulose matrix or a matrix containing a 1:1 combination of hydroxypropyl methylcellulose and carboxymethyl cellulose matrix. In one embodiment, the controlled-release quinine formulation comprises quinine as the only active agent. In one embodiment, the controlled-release quinine formulation is not in the form of a liposome. In another embodiment, the controlled-release quinine formulation does not contain lipid-encapsulated particles. In still yet another embodiment, the controlled-release quinine formulation is not a tablet comprising a coating prepared from latex aqueous dispersions of acrylic polymers e.g. Eudragit L 100-55, Eudragit L 100, or Eudragit S 100; or emulsion polymers Eudragit L 30D or Eudragit E 30D.

In one embodiment, the administration of a controlled-release quinine formulation to a patient causes the patient to experience a prolongation in the mean QT/QTc interval from baseline of less than about 20 ms, specifically less than about 10 ms, and more specifically less than about 5 ms.

In one embodiment, the therapeutically effective amount of quinine in the controlled-release formulation is an amount sufficient to significantly reduce the treated patient's risk of experiencing prolongation of the heart's QT interval or other adverse side effects as outline previously, while at the same time providing the desired therapeutic effect. A significant reduction is any detectable negative change that is statistically significant in a standard parametric test of statistical significance such as Student's T-test, where p<0.05.

The following examples further illustrate the invention but, of course, should not be construed as in any way limiting its scope.

### EXAMPLES

### Example 1

### Extended-Release Formulation of Quinine Sulfate, Dihydrate (cinchonan-9-ol, 6′-methoxy-, (8α,9R)-, Sulfate (2:1), Dihydrate)

### Example 1

| Ingredient | Weight (mg) | Weight (mg) |
|---|---|---|
| Quinine sulfate dihydrate | 490 | 650 |
| Carbopol 971P NF polymer | 460 | 500 |
| Lactose monohydrate | 40 | 40 |
| Talc | 5 | 5 |
| Lubricant | | |
| Magnesium Stearate | 5 | 5 |
| Total | 1000 | 1200 |

The ingredients excluding the lubricant are mixed in a high shear granulator. Water is added and the mixture wet granulated. The granulation is screened, dried, and milled. The granulation is added into a low shear blender, the lubricant is added, and blended. The final blend is compressed on a tablet press to form extended-release quinine dosage forms.

### Example 2

| Ingredient | Weight (mg) | Weight (mg) |
|---|---|---|
| Quinine sulfate dihydrate | 490 | 650 |
| Hydroxypropylmethylcellulose | 460 | 500 |
| Lactose monohydrate | 40 | 40 |
| Glidant | | |
| Colloidal Silicon Dioxide | 5 | 5 |
| Lubricant | | |
| Magnesium Stearate | 5 | 5 |
| Total | 1000 | 1200 |

The ingredients excluding the lubricant and glidant are mixed in a low shear blender for 20 minutes. The lubricant and glidant are added and blended for 5 minutes. The formulation is directly compressed on a tablet press.

### Example 3

| Ingredient | Weight (mg) | Weight (mg) |
|---|---|---|
| Quinine sulfate dihydrate | 490 | 650 |
| Hydroxypropylmethylcellulose | 200 | 250 |
| Hydroxyethylcellulose | 260 | 290 |
| Lactose monohydrate | 40 | 0 |
| Glidant | | |
| Colloidal Silicon Dioxide | 5 | 5 |
| Lubricant | | |
| Magnesium Stearate | 5 | 5 |
| Total | 1000 | 1200 |

The ingredients excluding the lubricant are mixed in a high shear granulator. Water and ethyl alcohol are added as a granulating solution and the mixture wet granulated. The granulation is screened, dried, and milled. The granulation is added into a low shear blender, the glidant and lubricant are added, and blended. The final blend is compressed on a tablet press.

### Example 4

### Pellets or Beads with a Modified-Release Coating

| Ingredient | Weight(g) |
|---|---|
| Quinine sulfate dihydrate | 9000 |
| Microcrystalline Cellulose | 800 |
| Lubricant | |
| Magnesium Stearate | 200 |
| Total | 10,000 |

The ingredients excluding the lubricant are mixed in a high shear blender for 10 minutes. The lubricant is added and blended for 3 minutes. The formulation is directly compressed into tablets or pellets or beads. The pellets or beads can also be manufactured by extrusion spheronization in which a wet mass of the composition is extruded alone or with the aid of fillers, glidants, or lubricants.

Modified Release Coating

| Ingredient | % |
|---|---|
| Methacrylic Acid Copolymer | 15 |
| Polyethylene Glycol 600 | 1 |
| Talc | 4 |
| Water/Ethyl Alcohol | 80 |
| | Removed in |
| | process |

Polyethylene glycol is added to a Water/Ethyl Alcohol dispersion of Methacrylic Acid Copolymer and mixed. Talc is added while stirring with a propeller mixer.

The pellets or beads are added into a perforated coating pan or a fluid bed with a Wurster insert. The coating is sprayed onto the pellets or beads. A coating level of about 5-20% coat weight is applied.

The coated pellet or beads are filled into capsule shells.

### Example 5

### Extended-Release Wax Formulation

| Ingredient | Weight(mg) | Weight (mg) |
|---|---|---|
| Quinine sulfate dihydrate | 490 | 650 |
| Carnauba Wax | 460 | 500 |
| Microcrystalline Cellulose | 40 | 40 |
| Glidant | | |
| Colloidal Silicon Dioxide | 5 | 5 |
| Lubricant | | |
| Magnesium Stearate | 5 | 5 |
| Total | 1000 | 1200 |

The ingredients excluding the lubricant and glidant are mixed in a high shear granulator. Water and ethyl alcohol are added and the mixture wet granulated. The granulation is screened, dried, and milled. The granulation is added into a low shear blender, the glidant and lubricant are added, and blended. The final blend is compressed on a tablet press.

### Example 6

### QTc Interval Measurements Following Single Doses of Quinine Sulfate

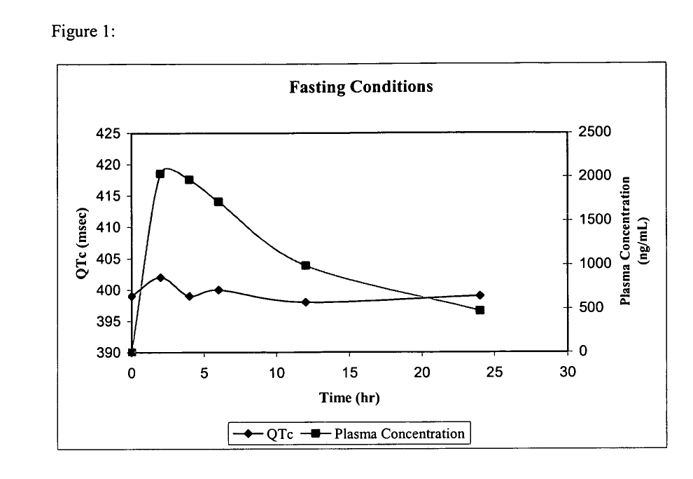

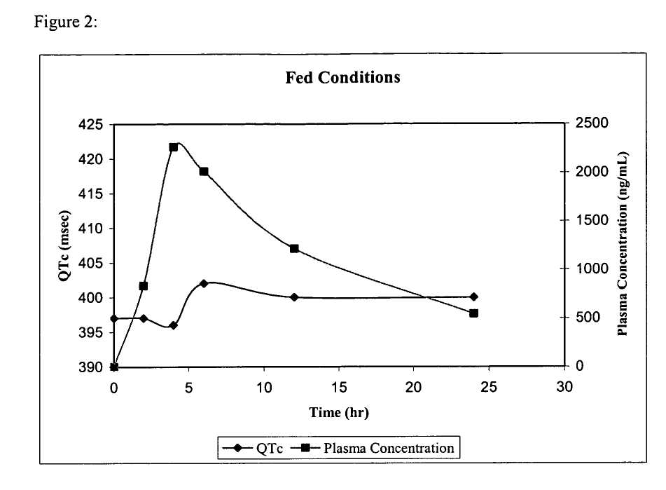

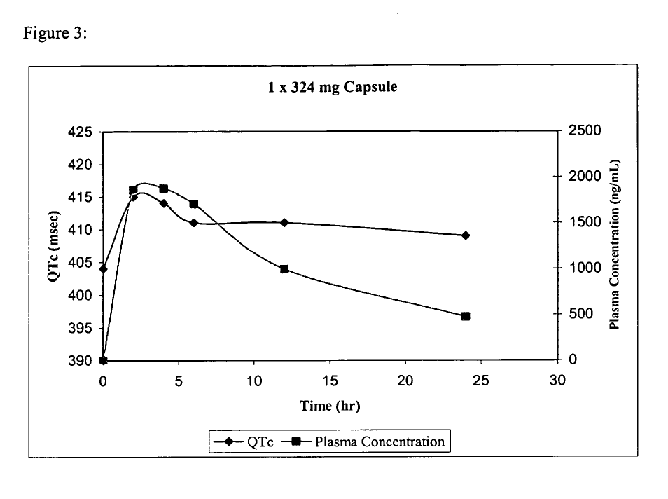

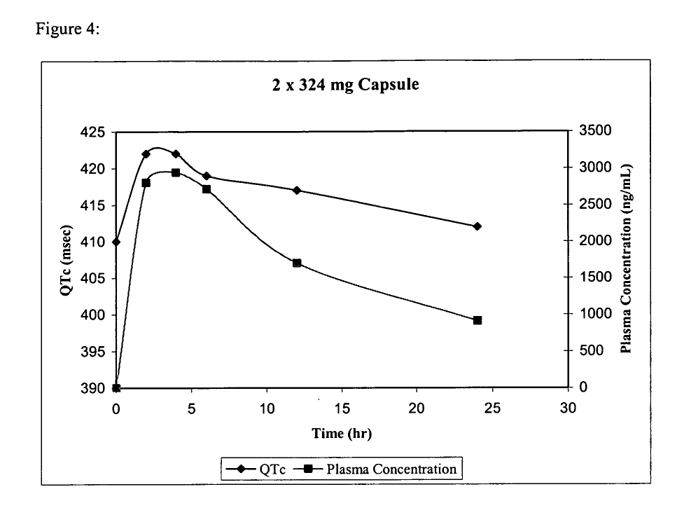

Studies were performed in healthy volunteers to measure QTc intervals following single doses of quinine sulfate. One study explored the effect of food on a single oral dose of a 324 mg oral capsule (324 mg quinine sulfate, 82 mg corn starch, 40 mg talc, 4 mg magnesium stearate). A second study was performed to compare two dose levels, a single oral dose of 324 mg quinine sulfate versus a single oral dose of 648 mg quinine sulfate (two capsules), both cases under fasting conditions. Repeated measurements of Electrocardiogram (ECG) intervals were taken for 50 subjects, 24 men and 26 women, who ranged in age from 18 to 47 years. The results are provided in Table 1 below and in FIGS. 1-4, which illustrate the correlation of mean maximum QTc interval prolongation effect to mean peak plasma quinine concentration.

TABLE 1

| | Study 1 | | Study 1 | | Study 2 | | Study 2 | |
|---|---|---|---|---|---|---|---|---|
| | A: 324 mg | | B: 324 mg | | C: 324 mg | | D: 648 mg | |
| | Quinine Sulfate | | Quinine Sulfate | | Quinine Sulfate | | Quinine Sulfate | |
| | capsule, Fasting | | capsule, Fed | | capsule, fasting | | capsules, fasting | |
| | conditions | | conditions | | conditions | | conditions | |
| Time | Mean Plasma Concentration (ng/ml); QTc (msec) | | | | | | | |
| (hours) | (ng/ml) | (msec) | (ng/ml) | (msec) | (ng/ml) | (msec) | (ng/ml) | (msec) |
| 0 | 0 | 399 | 0 | 397 | 0; | 404 | 0 | 410 |
| 2 | 2040 | 402 | 835 | 397 | 1860 | 415 | 2808 | 422 |
| 4 | 1971 | 399 | 2265 | 396 | 1877 | 414 | 2946 | 422 |
| 6 | 1718 | 400 | 2013 | 402 | 1707 | 411 | 2721 | 419 |
| 12 | 990 | 398 | 1216 | 400 | 994 | 411 | 1705 | 417 |
| 24 | 473 | 399 | 543 | 400 | 475 | 409 | 912 | 412 |

The data provided in columns A and B of Table 1 are directed to the mean plasma concentrations and QTc measurements over 24-hours following a single oral dose of a 324 mg Quinine Sulfate capsule under fasting (A) and fed (B) conditions. The data provided in column C of Table 1 are directed to the mean plasma concentrations and QTc measurements over 24-hours following a single oral dose of one 324 mg Quinine Sulfate capsule under fasting conditions. The data provided in column D of Table 1 are directed to the mean plasma concentrations and QTc measurements over 24-hours following a single oral dose of two 324 mg Quinine Sulfate capsule under fasting conditions.

As indicated by the data in the table, an increase in the mean QTc value was found to correspond with the peak quinine plasma concentration, which is reached in an average of 2.4 to 4.4 hours after oral administration in the fasted state and 4 to 6 hours when given with food was observed. Increases are higher when the same dose is given with food (which results in higher peak concentrations) and with a single dose of 648 mg as compared to 324 mg. In the study, seven subjects had significant prolongations in QTc interval (>450 msec). As illustrated, the higher the blood levels of quinine, the higher the incidence of QTc prolongation was observed. Not wishing to be bound by theory, but by leveling the blood level of quinine with little or no spiking of the blood plasma concentration, the incidence of QTc prolongation may be reduced or eliminated.

### Example 7

### Non-Linear Dose Proportionality Following Single Doses of Quinine Sulfate

A study was performed in healthy volunteers to measure the AUC (0-24 hours and 0-INF) and Cₘₐₓ following single oral doses of 1 and 2 capsules, each containing 324 mg quinine sulfate (324 mg quinine sulfate, 82 mg corn starch, 40 mg talc, 4 mg magnesium stearate per capsule), in the fasted state. The study was performed on 24 subjects. After administration of the doses, blood samples were taken from the subjects every half hour for the first four hours and then every hour up to 48 hours. The results were calculated as Ln-transformed data, geometric mean, as well as the least squares mean, non-transformed data. The geometric means are based on least squares means of ln-transformed values. The results, provided in Table 2a below, indicate that there is nonlinear dose proportionality where doubling the dose produces a Cₘₐₓ that is lower than would be expected with linear dose proportionality under fasted conditions. Cₘₐₓ resulted from multiplying plasma concentration by 2 for the 1 capsule treatment is summarized in Table 2a, and is 129% of that from the 2 capsule treatment with the 90% confidence interval from 122-138%. AUCT and AUC_{inf} showed proportional increase when given two capsules.

TABLE 2a

| | | | | | |
|---|---|---|---|---|---|
| | 324 mg | | | | |
| | Quinine Sulfate, | 648 mg | | 90% Confidence | |
| | 1 capsule* | Quinine | | Interval | P-values for |
| PK | (dose adjusted to | Sulfate, 2 | | (Lower Limit, | Product |
| variable | 2 × 324 mg) | capsules* | % Ratio | Upper Limit) | Effects |

Ln-transformed data, Geometric Mean

| | | | | | |
|---|---|---|---|---|---|
| Cₘₐₓ | 4126.31 | 3174.89 | 129.97 | (122.15, 138.29) | <0.0001 |
| (ng/ml) | | | | | |
| AUC₀₋ₜ | 61186.53 | 54440.26 | 112.39 | (106.56, 118.54) | 0.0011 |
| (ng- | | | | | |
| hr/ml) | | | | | |
| AUC_{0-INF} | 66715.41 | 59166.93 | 112.76 | (105.69, 120.3) | 0.0044 |
| (ng- | | | | | |
| hr/ml) | | | | | |

Non-transformed data, least squares mean

| | | | | | |
|---|---|---|---|---|---|
| Cmax | 4247.02 | 3243.11 | 130.96 | (123.28, 138.63) | <0.0001 |
| (ng/ml) | | | | | |
| AUC₀₋ₜ | 64277.02 | 56394.65 | 113.98 | (108.03, 119.93) | 0.0006 |
| (ng- | | | | | |
| hr/ml) | | | | | |
| AUC_{0-INF} | 70886.14 | 61817.27 | 114.67 | (107.37, 121.97) | 0.0023 |
| (ng- | | | | | |
| hr/ml) | | | | | |
| Tmax | 2.78 | 2.80 | 99.25 | (84.8, 113.7) | 0.9298 |
| kₑₗᵢₘ | 0.0592 | 0.0572 | 103.48 | (94.67, 112.28) | 0.5045 |
| t_{1/2} | 12.76 | 12.80 | 99.67 | (85.69, 113.66) | 0.9683 |

*The capsules contained quinine sulfate USP, corn starch, magnesium stearate, and talc.

### Example 8

### Dose Proportionality Following Single, Low Doses of Quinine Sulfate

A pediatric study was performed in healthy volunteers to measure the AUC (0-24 hours and 0-NF) and Cₘₐₓ following single oral doses of 260 mg quinine sulfate and 324 mg quinine sulfate (1.25 times the lower dose of 260 mg), in the fasted state. The study was performed on 22 subjects. After administration of the doses, blood samples were taken from the subjects every half hour for the first four hours, every hour up to 8 hours, and then at hours ten, twelve, sixteen, twenty-four, thirty-six, and forty-eight. The results were calculated as Ln-transformed data, geometric mean, as well as the least squares mean, non-transformed data. The geometric means are based on least squares means of Ln-transformed values. The results, provided in Table 2b below, indicate that there is linear dose proportionality when dosing quinine sulfate at the lower doses of 260 mg and 324 mg.

TABLE 2B

| | | | | | |
|---|---|---|---|---|---|
| | 260 mg | | | | |
| | Quinine Sulfate, | 324 mg | | 90% Confidence | |
| | 1 capsule* | Quinine | | Interval | P-values for |
| PK | (dose adjusted to | Sulfate, 1 | | (Lower Limit, | Product |
| variable | 1.25 × 324 mg) | capsules* | % Ratio | Upper Limit) | Effects |

Ln-transformed data, Geometric Mean

| | | | | | |
|---|---|---|---|---|---|
| Cₘₐₓ | 2251.55 | 2242.70 | 100.39 | (95.8, 105.21) | 0.8861 |
| (ng/ml) | | | | | |
| AUC₀₋ₜ | 30019.28 | 30318.55 | 99.01 | (93.83, 104.48) | 0.7535 |
| (ng-hr/ml) | | | | | |
| AUC_{0-INF} | 32072.92 | 32111.76 | 99.88 | (94.4, 105.67) | 0.9708 |
| (ng-hr/ml) | | | | | |

Non-transformed data, least squares mean

| | | | | | |
|---|---|---|---|---|---|
| Cmax | 2310.90 | 2275.46 | 101.56 | (95.93, 107.19) | 0.6384 |
| (ng/ml) | | | | | |
| AUC₀₋ₜ | 31285.26 | 31298.12 | 99.96 | (94.63, 105.28) | 0.9895 |
| (ng-hr/ml) | | | | | |
| AUC_{0-INF} | 33582.46 | 33280.89 | 100.91 | (95.36, 106.46) | 0.7811 |
| (ng-hr/ml) | | | | | |
| Tmax | 2.61 | 2.75 | 95.04 | (84.53, 105.55) | 0.4255 |
| kₑₗᵢₘ | 0.0615 | 0.0668 | 92.05 | (85, 99.04) | 0.0641 |
| t_{1/2} | 11.94 | 11.13 | 107.27 | (100.34, 114.2) | 0.0856 |

*The capsules contained quinine sulfate USP, corn starch, magnesium stearate, and talc.

Based on the results of Examples 7-8 illustrating non-dose proportionality for the higher dosage of quinine and the dose proportionality for the lower dosage, it is suggested that there is a need for controlled-release forms to achieve lower and more sustained plasma levels. By controlling the release of the quinine, sharp plasma peaks and troughs can be avoided thereby providing a safer profile for the administration of quinine.

The terms “a” and “an” do not denote a limitation of quantity, but rather denote the presence of at least one of the referenced item. The term “or” means “and/or”. The terms “comprising”, “having”, “including”, and “containing” are to be construed as open-ended terms (i.e., meaning “including, but not limited to”). The endpoints of all ranges directed to the same component or property are inclusive and independently combinable. All methods described herein can be performed in a suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention as used herein, the terms wt %, weight percent, percent by weight, etc. are equivalent and interchangeable.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. (canceled)

2. A controlled-release formulation, comprising:
a therapeutically effective amount of quinine and a release-retarding material;
wherein the release-retarding material is a release-retarding matrix, a release-retarding coating, or a combination comprising at least one of the foregoing; and
wherein dosing of the controlled-release formulation results in a reduction in severity or elimination of an adverse side effect associated with dosing of an immediate-release quinine formulation.

3. The formulation of claim 2, wherein the adverse side effect is cinchonism, tinnitus, blurred vision, thrombocytopenia, granulomatous hepatitis, skin rash, acute interstitial nephritis, thrombotic thrombocytopenia purpura-hemolytic-uremic syndrome (TTP-HUS), QT interval prolongation, QTc interval prolongation, agranulocytosis, hypoprothrombinemia, disseminated intravascular coagulation, hemolytic anemia, hemolytic uremic syndrome, headache, diplopia, confusion, altered mental status, seizures, coma, pruritus, flushing of the skin, sweating, occasional edema of the face, exanthema, urticaria, erythema multiforme, purpura, photosensitivity, contact dermatitis, acral necrosis, cutaneous vasculitis, asthmatic symptoms, tachycardia, irregular rhythm, premature ventricular contractions (PVCs), nodal escape beats followed the PVCs, U waves with normal PR, QRS, and QT intervals, ventricular fibrillation, arrhythmia, nausea and vomiting, abdominal pain, diarrhea, visual disturbances, including sudden loss of vision, blindness, diminished visual fields, fixed papillary dilatation, disturbed color vision, hearing loss, deafness, or a combination comprising at least one of the foregoing.

4. The formulation of claim 2, wherein the adverse side effect is QT interval prolongation or QTc interval prolongation.

5. The controlled-release formulation of claim 2, wherein dosing of the controlled-release formulation does not cause significant QT prolongation according to the standards of the United States Food and Drug Administration.

6. The formulation of claim 2, wherein the quinine is quinine sulfate; quinine sulfate, dihydrate; quinine hydrochloride; quinine dihydrochloride; or a combination comprising at least one of the foregoing.

7. (canceled)

8. The method of claim 36, wherein the release-retarding matrix is an acrylic or acrylate polymer, an acrylic or acrylate copolymer, an alkylcellulose, a shellac, a zein, a hydrogenated vegetable oil, a hydrogenated castor oil, a polyvinylpyrrolidine, a crosslinked polyvinylpyrrolidone, a vinyl acetate copolymer, a polyethylene oxide, a wax, a digestible long chain substituted or unsubstituted hydrocarbon, a fatty alcohol, a fatty acid, a fatty acid ester, a hydrogenated fat, a polymer or copolymer of lactic or glycolic acid, a polyalkylene glycol, a hydroxyalkylcellulose, a crosslinked hydroxyalkylcellulose, a carboxyalkylcellulose, a crosslinked carboxyalkylcellulose, a hydroxyalkyl alkylcellulose, a carboxyalkyl starch, a polyvinyl alcohol, a potassium methacrylate/divinylbenzene copolymer, or a combination comprising at least one of the foregoing release-retarding materials.

9. (canceled)

10. The method of claim 36, wherein the release-retarding coating is an alkylcellulose, a hydroxyalkylcellulose, a hydroxyalkyl alkylcellulose, a carboxyalkylcellulose, a carboxyalkyl alkylcellulose, a carboxyalkylcellulose ester, a starch, a polysaccharide, a carrageenan, a galactomannan, traganth, agar-agar, gum arabicum, guar gum, xanthan gum, an acrylic or acrylate polymer, polyvinylalcohol, polyvinylpyrrolidone, a copolymer of polyvinylpyrrolidone and vinyl acetate, a polyalkylene oxide, or a combination comprising at least one of the foregoing release-retarding coatings; and wherein the coating optionally further comprises a plasticizer.

11. (canceled)

12. The method of claim 36, wherein the controlled-release coating coats a granule, a particle, a tablet, a bead, or a combination comprising at least one of the foregoing.

13. (canceled)

14. The method of claim 36, wherein the controlled-release formulation is an oral dosage formulation, wherein the oral dosage formulation is a tablet, a capsule, a liquid, a suspension, an emulsion, an orally disintegrating tablet, a fast-dissolve tablet dosage formulation, a chewable tablet, a gastro-resistant tablet, a gastro-resistant capsule, an osmotic pump, or a combination comprising at least one of the foregoing.

15.-22. (canceled)

23. The controlled-release formulation of claim 2, wherein the controlled-release formulation provides therapeutically effective plasma levels for greater than about 16 hours after administration at steady state.

24. The method of claim 36, wherein the controlled-release formulation provides therapeutically effective plasma levels for greater than about 16 hours after administration at steady state.

25. The method of claim 36, wherein Tₘₐₓ of the controlled-release quinine formulation is about 1.5 to about 8 hours.

26. The method of claim 36, wherein the Cₘₐₓ is about 200 to about 7000 ng/mL and the Cₘᵢₙ is about 100 to about 3500 ng/mL at 12 to 24 hours, when at steady state.

27. The method of claim 36, wherein the duration of 50% or greater of Cₘₐₓ is about 10 to about 20 hours; or
wherein the duration of 80% or greater of Cₘₐₓ is about 2 to about 12 hours.

28.-29. (canceled)

30. The method of claim 36, wherein the formulation is prepared into a unit dosage form that exhibits a dissolution profile such that at 60 minutes after combining the dosage form with 900 ml of a dissolution medium at 37° C.±0.5° C. according to USP 28<711> test method 2 (paddle), 75 rpm paddle speed, about 10 to about 30 weight percent of the total amount of quinine is released, and wherein after 10 hours greater than or equal to about 70% of the total amount of quinine is released.

31. (canceled)

32. The method of claim 36, wherein the formulation is prepared into a unit dosage form that exhibits a dissolution profile such that at 60 minutes after combining the dosage form with 900 ml of purified water at 37° C.±0.5° C. according to USP 28<711> test method 2 (paddle), 75 rpm paddle speed, about 10 to about 30 weight percent of the total amount of quinine is released, and wherein after 10 hours greater than or equal to about 70% of the total amount of quinine is released.

33. (canceled)

34. The method of claim 36, wherein the formulation is prepared into a unit dosage form that exhibits a dissolution profile such that at 2 hours after combining the dosage form with 900 ml of a 0.1 N Hydrochloric Acid medium at 37° C.±0.5° C. according to USP 28 <711> test method 1 or 2, about 0 to about 50 weight percent of the total amount of quinine is released and wherein after 2 hours when the medium is switched to a buffer phase of pH 4.5, 6.8, 7.0 or water, about 0 to about 100 weight percent of the total amount of quinine is released.

35. (canceled)

36. A method of treating a patient, comprising administering a controlled-release quinine formulation to a patient, wherein the controlled-release formulation comprises
a therapeutically effective amount of quinine and a release-retarding material;
wherein the release-retarding material is a release-retarding matrix, a release-retarding coating, or a combination comprising at least one of the foregoing; and
wherein the controlled-release formulation provides therapeutically effective plasma levels for greater than about 12 hours after administration at steady state.

37. (canceled)

38. A controlled-release formulation, comprising:
a therapeutically effective amount of quinine and a release-retarding material;
wherein the release-retarding material is a release-retarding matrix, a release-retarding coating, or a combination comprising at least one of the foregoing; and
wherein the release-retarding matrix is an alkylcellulose, a shellac, a zein, a hydrogenated vegetable oil, a hydrogenated castor oil, a polyvinylpyrrolidine, a crosslinked polyvinylpyrrolidone, a vinyl acetate copolymer, a wax, a digestible long chain substituted or unsubstituted hydrocarbon, a fatty alcohol, a fatty acid, a fatty acid ester, a hydrogenated fat, a crosslinked hydroxyalkylcellulose, a polyvinyl alcohol, or a combination comprising at least one of the foregoing release-retarding materials; and
wherein the release-retarding coating is alkylcellulose, a hydroxyalkylcellulose, a hydroxyalkyl alkylcellulose, a starch, a polysaccharide, agar-agar, gum arabicum, guar gum, xanthan gum, polyvinylalcohol, polyvinylpyrrolidone, a copolymer of polyvinylpyrrolidone and vinyl acetate, a polyalkylene oxide, or a combination comprising at least one of the foregoing release-retarding coatings; and wherein the coating optionally further comprises a plasticizer.

39. The formulation of claim 38, wherein the matrix further comprises a polyethylene oxide, a polyalkylene glycol, an acrylic or acrylate polymer, an acrylic or acrylate copolymer, a polymer or co-polymer of lactic or glycolic acid, a crosslinked carboxyalkylcellulose, a carboxyalkyl starch, a potassium methacrylate/divinylbenzene copolymer, a carboxyalkylcellulose, a hydroxyalkyl alkylcellulose, a hydroxyalkylcellulose, or a combination comprising at least one of the forgoing;
wherein the release-retarding coating further comprises an acrylic or acrylate polymer, a carboxyalkylcellulose, a carboxyalkyl alkylcellulose, a carboxyalkylcellulose ester, a carrageenan, a galactomannan, traganth, or a combination comprising at least one of the forgoing

40. (canceled)
